SEQUENCE LISTING (10 sequences)

SEQ ID NO: 1 - protein, 5 aa, Artificial sequence
  Levilimab_HCDR1
SYYMS

SEQ ID NO: 2 - protein, 16 aa, Artificial sequence
  Levilimab_HCDR2
GIYSDGTTHYGDSVKG

SEQ ID NO: 3 - protein, 12 aa, Artificial sequence
  Levilimab_HCDR3
GAGPTWWYALDA

SEQ ID NO: 4 - protein, 120 aa, Artificial sequence
  Levilimab_VH
QVQLVQSGGGLVQPGGSLRLSCAASGFTFSSYYMSWVRQAPGKGLEWVSGIYSDGTTHYGDSVKGRFTISRDNAKNTVYLQLNSLRAEDTAMYYCAKGAGPTWWYALDAWGQGTLVTVSS

SEQ ID NO: 5 - protein, 450 aa, Artificial sequence
  Levilimab_HC
QVQLVQSGGGLVQPGGSLRLSCAASGFTFSSYYMSWVRQAPGKGLEWVSGIYSDGTTHYGDSVKGRFTISRDNAKNTVYLQLNSLRAEDTAMYYCAKGAGPTWWYALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 15 aa, Artificial sequence
  Levilimab_LCDR1
KSSQSVLSASNTYLN

SEQ ID NO: 7 - protein, 7 aa, Artificial sequence
  Levilimab_LCDR2
YASTRES

SEQ ID NO: 8 - protein, 9 aa, Artificial sequence
  Levilimab_LCDR3
QQAYRAPVT

SEQ ID NO: 9 - protein, 111 aa, Artificial sequence
  Levilimab_VL
DIQLTQSPSSVSVSVGERVTIDCKSSQSVLSASNTYLNWYQQKPGQAPQLLIYYASTRESGVPDRFSGSGSGTDFTLTISSLQAEDAAVYYCQQAYRAPVTFGQGTKLEIK

SEQ ID NO: 10 - protein, 218 aa, Artificial sequence
  Levilimab_LC
DIQLTQSPSSVSVSVGERVTIDCKSSQSVLSASNTYLNWYQQKPGQAPQLLIYYASTRESGVPDRFSGSGSGTDFTLTISSLQAEDAAVYYCQQAYRAPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0203171 A1
(43) Pub. Date: Jun. 29, 2023

(54) AQUEOUS PHARMACEUTICAL COMPOSITION OF LEVILIMAB

(71) Applicant: JOINT STOCK COMPANY "BIOCAD", Saint Petersburg (RU)

(72) Inventors: Dmitrii Aleksandrovich TOLSTYKH, G. Ekaterinburg (RU); Alina Aleksandrovna TSUKUR, Saint-Petersburg (RU); Ekaterina Aleksandrovna LOMKOVA, Saint Petersburg (RU); Aleksandr Olegovich IAKOVLEV, Moscow (RU); Anton Aleksandrovich LUTCKII, Saint-Petersburg (RU); Iuliia Nikolaevna LINKOVA, Saint-Petersburg (RU); Arina Valerevna ZINKINA-ORIKHAN, Saint-Petersburg (RU); Dmitry Valentinovich MOROZOV, Saint-Petersburg (RU)

(21) Appl. No.: 17/998,567

(22) PCT Filed: Jun. 7, 2021
(86) PCT No.: PCT/RU2021/050158

§ 371 Date: Nov. 11, 2022

(30) Foreign Application Priority Data
Jun. 5, 2020 (RU) 2020118737

(51) Int. Cl. C07K 16/28; A61K 47/12; A61K 47/10; A61K 47/18; A61K 31/519; A61K 9/08; A61P 19/02; A61P 37/06

(52) U.S. Cl. CPC C07K 16/2866; A61K 47/12; A61K 47/10; A61K 47/183; A61K 31/519; A61K 9/08; A61P 19/02; A61P 37/06; C07K 2317/21

(57) ABSTRACT

The disclosure relates to the field of pharmacy and medicine, specifically to aqueous compositions of anti-IL-6R antibody levilimab which may be used as a medicinal product for treating IL-6R-associated diseases.

### FIELD OF THE INVENTION

The present invention relates to the field of pharmacy and medicine, specifically to aqueous compositions of anti-IL-6R antibody levilimab, which may be used as a medicinal product for treating IL-6R-associated diseases.

### BACKGROUND OF THE INVENTION

Interleukin-6 (IL-6, IL6) is one of the main pro-inflammatory cytokines. IL-6 is produced by activated monocytes, macrophages, T cells, as well as some other cells. Along with other cytokines it is involved in processes related to immune response, inflammation, angiogenesis, bone metabolism. The main effect of IL-6 is associated with its participation in the differentiation of B lymphocytes, their maturation and transformation into plasma cells secreting immunoglobulins. Furthermore, IL-6 induces the expression of the IL-6 receptor on activated cells of the immune system, and further induces the production of IL-2 by T lymphocytes. IL-6 stimulates the proliferation of T lymphocytes and hematopoiesis responses. In terms of variety of producer cells and targets for biological effects, interleukin-6 is one of the most active cytokines involved in realization of immune and inflammatory responses. It was shown that the disbalance between pro- and antiinflammatory effects of IL-6 results in various autoimmune diseases; chronic inflammation and osteoporosis, psoriasis, while its excessive production is associated with various forms of cancer.

Thus, the inhibition of IL-6 is an attractive therapeutic target (Peter C. HeinrichBiochem. J. (2003) 374:1).

When activated, the IL-6 receptor (IL-6R, IL6R) triggers a cascade of reactions in the cell, which lead to the active synthesis of proteins involved in the inflammatory response. The receptor is activated during binding of IL-6 to the IL-6 (CD126) receptor subunit alpha, and two gp130 molecules transducing a signal inside the cell (Simon A. Jones The FASEB Journal 15(1): 43-58). There are 2 forms of the α-receptor: membrane (mIL-6R) and soluble (sIL-6R). The soluble form is produced as a result of proteolysis of the transmembrane portion mIL-6R or alternative splicing of mIL-6R mRNA. The soluble form sIL-6R provides a response to IL-6 cells without surface mIL-6R.

Thus, the IL-6 signal is transduced into a cell by two pathways. The first pathway (classical signaling), in which IL-6 binds to immune system cells expressing on their surface mIL-6R associated with a gp130 molecule. In the second pathway (trans-signaling), IL-6 binds to a circulating sIL-6R to form a complex that binds to the cells having only gp130 molecules on their membrane, i.e., potentially to any cells of the human body. In this case, the complete IL-6 receptor complex is assembled on the cell membrane, which is followed by the induction of a signaling cascade in the cell.

Blocking the effect of IL-6 and, therefore, inflammatory reaction may be achieved by preventing the complete assembly of the IL-6 receptor complex consisting of an alpha-subunit, gp130 molecules, and IL-6. When binding to IL-6R, polypeptides are able to interfere with the assembly of the complete complex; accordingly, they block the signal transduction into the cell.

The polypeptides that specifically bind to IL-6 (patent RU2550262), IL-6R, or gp130 have exhibited a significant inhibitory effect on the functioning of IL-6. An antibody binding to IL-6R, tocilizumab, is currently known, which is a recombinant humanized monoclonal antibody of the IgG1ₖ (gamma-1, kappa) immunoglobulin subclass, constructed by grafting the complementarity-determining region (CDR) of a murine anti-IL-6R antibody onto human IgG1.

Medicinal products based on the antibody (tocilizumab) which binds to IL-6R and blocks its interaction with IL-6 are used in treatment of rheumatoid arthritis and systemic juvenile idiopathic arthritis both as monotherapy, and in combination with methotrexate and/or other basic anti-inflammatory drugs.

Further known is a novel antibody to IL-6R, levilimab (also known as BCD-089), which is IgG1 isotype monoclonal antibody with mutations introduced into the constant portion. Levilimab is currently undergoing clinical trials in patients with various diseases, including rheumatoid arthritis and (acute) respiratory distress syndrome in adults.

It is known that the use of monoclonal antibodies against the interleukin-6 receptor (IL6R, IL-6R) can effectively reverse the cytokine storm syndrome that develops when using CAR T therapy in cancer treatment. The efficacy of therapy (reversing the syndrome within 14 days from the first and only administration) reaches 69%.

In the context of the COVID-19 pandemic, the successful use of anti-IL-6R therapy in patients with severe or critical cases of COVID-19 pneumonia has been shown. A meta-analysis of published data on the efficacy of IL-6R inhibitors in patients with COVID-19 preliminarily confirmed their efficacy (Xu, X.; Han, M.; etc., Effective treatment of severe COVID-19 patients with tocilizumab. Proc. Natl. Acad. Sci. USA 2020; Coomes, E. A.; Haghbayan, H., Interleukin-6 in COVID-19: A Systematic Review and Meta-Analysis. medRxiv 2020, 2020.03.30.20048058).

In the clinical course of COVID-19 pneumonia, there is a window period between diagnosis and the development of multiple organ failure syndrome, which is about 5-7 days, following which most patients show improvement, but about 20% of patients show an increase in the severity of pneumonia (CRS, ARDS). To improve the prognosis and reduce mortality, it is recommended to use proactive anti-inflammatory therapy starting from the moment of diagnosis of COVID-19 pneumonia (Sun, X.; Wang, T.; etc., Cytokine storm intervention in the early stages of COVID-19 pneumonia. Cytokine Growth Factor Rev 2020).

The IL-6R inhibitors have been included in the Russian COVID-19 treatment guidelines as drugs of proactive anti-inflammatory treatment of COVID-19 in adults (for patients with moderate to severe course: with acute respiratory distress syndrome, cytokine storm syndrome).

It follows from the above that there is currently a need for the creation of novel improved stable aqueous pharmaceutical compositions for the anti-IL-6R antibody levilimab.

### BRIEF DESCRIPTION OF DRAWINGS

FIG. 1 is a graph of dependence of the optical density of solutions at 400 nm on PEG concentration for the monoclonal antibody against the IL-6 receptor levilimab in the test formulations.

FIG. 2 is a graph illustrating the temperature trend of the pharmaceutical composition 5 Acet.Buf+300Glu (selection of osmotic agent).

FIG. 3 is a graph illustrating the temperature trend of the pharmaceutical composition 5 Acet Buf.+Mann (selection of osmotic agent).

FIG. 4 is a graph illustrating the temperature trend of the pharmaceutical composition 5 Acet. Buf+100Arg+Mann (selection of osmotic agent).

FIG. 5 is a graph illustrating the temperature trend of the pharmaceutical composition 5 Acet Buf.+200Arg (selection of osmotic agent).

FIG. 6 is a graph illustrating the change in quality indicators as a function of time under accelerated storage condition at a concentration of levilimab of 220 mg/ml.

FIG. 7 is a graph illustrating the change in quality indicators as a function of time at accelerated storage condition at a concentration of levilimab of 180 mg/ml.

FIG. 8 is a graph illustrating the change in quality indicators as a function of time under accelerated storage condition at a concentration of levilimab of 20 mg/ml.

FIG. 9 is a graph illustrating the proportion of patients who achieved improvement in the course of the disease, corresponding to ACR20 by week 4, 8, 12, 16, 24, 36, 48, and 52.

FIG. 10 is a graph illustrating the proportion of patients who achieved improvement in the course of the disease, corresponding to ACR50 by week 4, 8, 12, 16, 24, 36, 48, and 52.

FIG. 11 is a graph illustrating the proportion of patients who achieved improvement in the course of the disease, corresponding to ACR70 by week 4, 8, 12, 16, 24, 36, 48, and 52.

FIG. 12 is a graph illustrating the change in the DAS-28-CRP index relative to the baseline over the course of 52 weeks of treatment.

FIG. 13 is a graph illustrating the proportion of patients who achieved remission of the disease at week 24, 36, 48, and 52 of treatment.

FIG. 14 is a graph illustrating changes in ESR with treatment.

FIG. 15 is a graph illustrating the dynamics of the concentration of the soluble interleukin-6 receptor in patients throughout 12 weeks of treatment.

FIG. 16 is a graph illustrating the change in the concentration of C-reactive protein in patients' serum throughout 12 weeks of treatment.

### DESCRIPTION OF THE INVENTION

### Definitions

Unless defined otherwise herein, all technical and scientific terms used in connection with the present invention shall have the same meaning as is commonly understood by those skilled in the art.

Further, unless otherwise required by context, singular terms shall include pluralities and plural terms shall include the singular terms. Typically, the present classification and methods of cell culture, molecular biology, immunology, microbiology, genetics, analytical chemistry, organic synthesis chemistry, medical and pharmaceutical chemistry, as well as hybridization and chemistry of protein and nucleic acids described herein are well known by those skilled and widely used in the art. Enzyme reactions and purification methods are performed according to the manufacturer's guidelines, as is common in the art, or as described herein.

The term “antibody” or “immunoglobulin” (Ig) as used in this description includes full-size antibodies and any antigen binding fragment (i.e., “antigen-binding portion”) or separate chains thereof.

The term “antigen-binding portion” of an antibody or “antigen-binding fragment” (or simply “antibody portion” or “antibody fragment”), as used in this description, refers to one or more fragments of an antibody that retain the capability of specific binding to an antigen. It has been shown that the antigen-binding function of an antibody can be performed by the fragments of a full-size antibody. Examples of binding fragments which are included within the term “antigen-binding portion” of an antibody include (i) Fab-fragment, a monovalent fragment, consisting of the VL, VH, CL and CH 1 domains; (ii) F(ab′)2 fragment, a bivalent fragment, comprising two Fab-fragments linked by a disulfide bridge at the hinge region; (iii) Fd-fragment consisting of the VH and CH1 domains; (iv) Fv-fragment consisting of the VL and VE domains in a single arm of an antibody; (v) dAb-fragment (Ward et al., (1989) Nature 341:544-546), which consists of the VH/VHH domain; and (vi) an isolated complementarity determining region (CDR). In addition, two regions of the Fv-fragment, VL and VH, are encoded by different genes, they can be joined using recombinant methods using a synthetic linker that enables to receive them as a single protein chain in which the VL and VH regions are paired to form monovalent molecules (known as a single-chain Fv (scFv); see e.g. Bird et al. (1988) Science 242:423-426; and Huston et al. (1988) Proc. Natl. Acad. Sci. USA 85:5879-5883). It is assumed that such single-stranded molecules are also included within the term “antigen-binding portion” of antibody. Such antibody fragments are produced using conventional techniques known to those skilled in the art, and these fragments are screened in the same manner as intact antibodies are.

Preferably, the CDR of an antigen-binding portion, or the whole antigen binding portion of antibodies of the invention is derived from a mouse, lama, or human donor library or substantially of human origin with certain amino acid residues altered, e.g., substituted with different amino acid residues so as to optimize specific properties of the antibody, e.g., KD, koff, IC50, EC50, ED50. Preferably, the framework regions of the antibody of the invention are of human origin or substantially of human origin (at least 80, 85, 90, 95, 96, 97, 98 or 99% of human origin).

The term “monoclonal antibody” or “mAb” refers to an antibody that is synthesized and isolated by a separate clonal population of cells. The clonal population may be a clonal population of immortalized cells. In some embodiments of the invention, the immortalized cells within the clonal population are hybrid cells, hybridomas, typically produced by the fusion of individual B lymphocytes from immunized animals with individual cells of a lymphocytic tumor. Hybridomas are a type of constructed cells and do not exist in nature.

A population of “monoclonal antibodies” as used herein refers to a homogenous or substantially homogeneous antibody population (i.e., at least about 96%, but more preferably at least about 97 or 98%, or yet more preferably at least 99% of antibodies within the population will compete for the same antigen or epitope in the enzyme-linked immunosorbent assay ELISA, or more preferably the antibodies are identical in the amino acid sequence).

A naturally-occurring full-size antibody is an immunoglobulin molecule that consists of four polypeptide chains (two heavy (H) chains (of about 50-70 KDa for the full length), and two light (L) chains (of about 25 KDa for the full length) linked by disulfide bonds. The amino-terminal portion of each chain includes a variable domain of about 100-110 or more amino acids that are responsible for binding an antigen. The carboxyl-terminal portion of each chain determines the constant region that is mostly responsible for the effector function. Light chains are classified as kappa and lambda and characterized by a specific constant region. Each light chain consists of a variable N-terminal light chain region (in this application referred to as VL or VK) and a constant light chain region that consists of a single domain (CL or CK). Heavy chains are classified as γ, δ, α, μ, and ε and define the antibody isotype, such as IgG, IgM, IgA, IgD and IgE, respectively; and some of them can be further divided into sub-classes (isotypes), such as IgG1, IgG2, IgG3, IgG4, IgA1 and IgA2. Each heavy chain type is characterized by a particular constant region Fc. Each heavy chain consists of a variable N-terminal heavy chain region (in this application referred to as VH) and constant (heavy chain) region CH. The constant heavy chain region consists of three domains (CH1, CH2 and CH3) for IgG, IgD and IgA, and of 4 domains (CH1, CH2, CH3 and CH4) for IgM and IgE. VH and VL variable domains may further be divided into the regions of hypervariability (hypervariable regions, CDRs) interspersing with more conservative framework regions (FRs). Each variable domain consists of three CDRs and four FRs located in the following order from N-terminus to C-terminus: FR1, CDR1, FR2, CDR2, FR3, CDR3 and FR4.

The variable regions of each of light/heavy chain pairs form the antigen-binding sites of an antibody. Thus, an intact IgG antibody has two binding sites. Except for bi-functional or bi-specific antibodies, the two binding sites are identical. As used in this application, “antigen-binding portion” or “antigen-binding region”, or “antigen-binding domain”, are interchangeable with reference to such an antibody molecule portion that comprises the amino acid residues interacting with an antigen and giving the antibody its specificity and affinity in relation to the antigen. Such antibody portion includes framework amino acid residues necessary for maintaining the proper conformation of antigen-binding residues.

“Antibody fragment” may be represented by an antibody fragment or antibody fragment that has the activity of a full-size antibody. Said antibody fragment may be F(ab′)2, F(ab)2, Fab′, Fab Fv and scFv.

The term “inhibits” or “neutralize”, as used in this application, with regard to the functional activity of the antibody of the invention refers to the ability to significantly block, prevent, restrict, slow down, stop, reduce, or reverse, for example, the development or severity of inhibition subject, including but not limited to biological activity (for example, the activity of IL-6R) or property, disease, or condition. Binding of the antibody of the invention with IL-6R results in the inhibition or neutralization of IL-6R activity preferably of at least 20, 30, 40, 50, 60, 70, 80, 90, 95% or higher.

The term “separated” or “isolated” when used with regard to a nucleic acid or protein product (for example, an antibody) refers to a nucleic acid molecule or protein molecule that is identified and separated from at least one contaminating substance to which it is typically combined in the natural source. Preferably, an “isolated antibody” is an antibody that substantially contains no other antibodies having a distinctive antigenic specificity (for example, pharmaceutical compositions, according to the present invention, comprise an isolated antibody that specifically binds IL-6R and substantially contains no antibodies that specifically bind antigens other than IL-6R).

The term “specifically binds” as used in this application refers to the situation in which one member of a specific binding pair does not significantly bind to molecules other than the specific binding partner(s) thereof. This term also applies if, for example, an antigen-binding domain of the antibody of the invention is specific for particular epitope that is carried by a number of antigens; in this case, the specific antibody having an antigen-binding domain will be capable of specific binding of various epitope-carrying antigens.

“Kabat numbering scheme” or “numbering according to Kabat” as used in this application refers to the system for numbering of amino acid residues that are more variable (i.e. hypervariable) than other amino acid residues in variable regions of heavy and light chains of the antibody (Kabat et al. Ann. N.Y. Acad. Sci., 190:382-93 (1971); Kabat et al. Sequences of Proteins of Immunological Interest, Fifth Edition, U.S. Department of Health and Human Services, NIH Publication No. 91-3242 (1991)).

The term “pharmaceutical composition” refers to a composition and/or formulation containing a therapeutically effective amount of the antibody according to the invention plus excipients or auxiliary substances (carriers, diluents, vehicles, solvents, and other excipients).

The term “buffer” or “buffer solution” refers to an aqueous solution comprising a mixture of an acid (typically a weak acid, such as, e.g. acetic acid, citric acid) and a conjugate base thereof (such as e.g. an acetate or citrate salt, e.g. sodium acetate, sodium citrate, as well as hydrates of said salts, e.g. sodium acetate trihydrate) or alternatively a mixture of a base (typically a weak base, e.g. histidine) and conjugate acid thereof (e.g. histidine hydrochloride). The pH value of a “buffer solution” changes only slightly upon addition thereto of a small quantity of strong base or strong acid, as well as upon dilution or concentration due to the “buffering effect” imparted by the “buffering agent”.

In this application, a “buffer system” comprises one or more buffering agent(s) and/or an acid/base conjugate(s) thereof, and more suitably comprises one or more buffering agent(s) and acid/base conjugate(s) thereof, and most suitably comprises only one buffering agent and an acid/base conjugate thereof. Unless specified otherwise, any concentrations used in the present invention in relation to a “buffer system” (buffer concentration) may refer to the combined concentration of buffering agent(s) and/or acid/base conjugate(s) thereof. In other words, concentrations used in this application in relation to a “buffer system” may refer to the combined concentration of the relevant buffering species (i.e., the species in dynamic equilibrium with one another, e.g., citrate/citric acid). The overall pH value of the composition comprising the relevant buffer system is a reflection of the equilibrium concentration of each of the relevant buffering species (i.e., the balance of buffering agent(s) to acid/base conjugate(s) thereof).

The term “buffering agent” refers to an acid or base component (typically a weak acid or weak base) of the buffer or buffer solution. The buffering agent helps to maintain the pH value of a given solution at or near to a pre-determined value, and the buffering agents are generally chosen to complement the pre-determined value. The buffering agent may be a single compound which gives rise to a desired buffering effect, especially when said buffering agent is mixed with (and suitably capable of proton exchange with) an appropriate amount (depending on the pre-determined value desired) of its corresponding “acid/base conjugate”.

As used herein, the term “solubilizer” refers to a pharmaceutically acceptable non-ionic surfactant. Both one solubilizer and combinations of solubilizers may be used. Exemplary solubilizers are, without limitation, polysorbate 20 or polysorbate 80, poloxamer 184 or poloxamer 188, or PLURONIC®.

The terms “osmotic agent” or “tonicity-regulating agent”, as well as “osmolyte”, as used herein, refer to an excipient that can provide the required osmotic pressure of a liquid antibody solution. In some embodiments, the tonicity-regulating agent may increase the osmotic pressure of a liquid antibody product to isotonic pressure such that said liquid antibody product is physiologically compatible with the cells of the tissue of a subject's organism. In another embodiment, the tonicity-regulating agent may contribute to increased stability of antibodies. “Isotonic” medicine is a medicine that has an osmotic pressure equivalent to that of human blood. Isotonic formulations typically have an osmotic pressure from about 239 to 376 mOsm/kg. The term “hypotonic” describes a formulation with an osmotic pressure below that of human blood. Correspondingly, the term “hypertonic” is used to describe a formulation with an osmotic pressure above that of human blood. Isotonicity may be measured using, e.g., a vapor pressure or cryoscopic osmometer. The tonicity-regulating agent may be present in an enantiomeric (e.g., L- or D-enantiomer) or racemic form; in the form of isomers such as alpha or beta, including alpha, alpha; or beta, beta; or alpha, beta; or beta, alpha; in the form of a free acid or free base; in the form of a salt; in a hydrated form (e.g., monohydrate), or in an anhydrous form. Exemplary osmotic agents are, but not limited to, sugars (trehalose dihydrate, sucrose, glucose), polyols (mannitol, sorbitol), amino acids (proline, arginine, glycine), or salts (sodium chloride, potassium chloride, magnesium chloride).

The term “long-term storage” or “long term stability” is understood to mean that a pharmaceutical composition may be stored for three months or more, for six months or more, and preferably for one year or more, most preferably with a minimum stable shelf life of at least two years. In general, the terms “long term storage” and “long term stability” further include stable storage durations that are at least comparable to or better than the stable shelf life typically required for currently available commercial formulations of the anti-IL-6R antibody levilimab, without losses in stability that would render the formulation unsuitable for its intended pharmaceutical application.

The term “parenteral administration” refers to administration regimens, typically performed by injection (infusion), and includes, in particular intravenous, intramuscular, intraarterial, intratracheal, intracapsular, intraorbital, intracardiac, intradermal, intraperitoneal, transtracheal, subcutaneous, intraarticular, subcapsular, subarachnoid, intraspinal, epidural and epigastric injection or infusion

The term “medicament” or “formulation” is a substance (or a mixture of substances as a pharmaceutical composition) in the form of tablets, capsules, solutions, ointments, and other ready forms intended for restoration, improvement, or modification of physiological functions in humans and animals, and for treatment and prophylaxis of diseases, for diagnostics, anesthesia, contraception, cosmetology, and others.

The term “IL-6R-associated disease or disorder” or “IL-6R-mediated disease or disorder” refers to all diseases or disorders that are either directly or indirectly related to IL6 signaling pathway activation, including the etiology, pathogenesis, progression, persistence, or pathology of the disease or disorder.

The term “use” applies to the possibility to use the antibody of the present invention or a pharmaceutical composition containing thereof to treat, relief the course of the disease, expedite the remission, reduce the recurrence rate for the diseases or disorders mediated by receptors with which the antibody of the present invention can bind. Exemplary diseases are but not limited to rheumatoid arthritis, juvenile chronic arthritis, scleroderma, graft versus host disease, organ transplant rejection, acute or chronic immune disease associated with organ transplantation, cachexia, adult (acute) respiratory distress syndrome, Still's disease, systemic scleroderma, Sjogren's syndrome, Takayasu's disease/arteritis, cytokine therapy associated disorders, cytokine release syndrome, iridocyclitis, uveitis, optic neuritis, optical neuromyelitis, juvenile rheumatoid arthritis, giant cell arteritis, polyarticular juvenile idiopathic arthritis, systemic-onset juvenile idiopathic arthritis; cancer, in particular multiple myeloma and malignant solid tumors, colorectal cancer, prostate cancer, ovarian cancer.

The term “method of treatment” refers to the possibility to use the antibody of the invention or a pharmaceutical composition containing thereof to treat, relief the course of the disease, expedite the remission, reduce the recurrence rate following the diseases or disorders associated with IL-6R activity. “Treat” or “treatment”, “prophylaxis” of a disease, disorder or condition may comprise the prevention or delay of the onset of clinical symptoms of a disease, disorder or condition developing in human, the inhibition of a disease, disorder or condition, i.e. stop, reduction or delay of the development of a disease or a relapse thereof (in case of maintenance therapy) or at least one clinical or subclinical symptom thereof, or the alleviation or easement of a disease, i.e. the causing of regression of a disease, disorder or condition. Exemplary diseases are but not limited to rheumatoid arthritis, juvenile chronic arthritis, scleroderma, graft versus host disease, organ transplant rejection, acute or chronic immune disease associated with organ transplantation, cachexia, adult (acute) respiratory distress syndrome, Still's disease, systemic scleroderma, Sjogren's syndrome, Takayasu's disease/arteritis, cytokine therapy associated disorders, cytokine release syndrome, iridocyclitis, uveitis, optic neuritis, optical neuromyelitis, juvenile rheumatoid arthritis, giant cell arteritis, polyarticular juvenile idiopathic arthritis, systemic-onset juvenile idiopathic arthritis; cancer, in particular multiple myeloma and malignant solid tumors, colorectal cancer, prostate cancer, ovarian cancer.

The term “aqueous composition” as used herein refers to a water-based composition, the water in the composition may be: water, water for injections, physiologic saline (0.9%-1.0% aqueous solution of sodium chloride).

In one embodiment of the invention, the subject of treatment, or patient, is a mammal, preferably a human subject. Said subject may be either male or female, of any age.

As used in the present description and claims that follow, unless otherwise dictated by the context, the words “have,” “include,” “comprise,” or their variations such as “has,” “having,” “includes” or “including”, “comprises,” “comprising,” shall be understood to imply the inclusion of the mentioned integer or group of integers but not the exclusion of any other integer or group of integers.

### SUMMARY OF THE INVENTION

The present invention discloses stable aqueous pharmaceutical compositions for the anti-IL-6R antibody levilimab, which can be used as a medicinal product for the treatment of IL-6R-associated diseases.

The antibody to IL-6R levilimab, which is IgG1 isotype monoclonal antibody, includes a heavy chain (HC) with the amino acid sequence of SEQ ID NO: 5, where the heavy chain variable domain (SEQ ID NO: 4) comprises HCDR1 (SEQ ID NO: 1), HCDR2 (SEQ ID NO: 2), and HCDR3 (SEQ ID NO: 3); and a light chain (LC) with the amino acid sequence of SEQ ID NO: 10, where the light chain variable domain (SEQ ID NO: 9) comprises LCDR1 (SEQ ID NO: 6), LCDR2 (SEQ ID NO: 7), and LCDR3 (SEQ ID NO: 8).

Levilimab is a recombinant monoclonal antibody to the interleukin-6 receptor. Levilimab binds to and blocks both soluble (sIL-6R) and membrane (mIL-6R) IL-6 receptors. Blockade of the both receptor forms prevent the development of the IL-6-associated pro-inflammatory cascade, including activation of antigen-presenting cells, B- and T cells, monocytes and macrophages, endothelial cells and fibroblasts, and excessive production of other pro-inflammatory cytokines. IL-6 is involved in the activation and maintenance of local inflammatory responses (formation of pannus in synovia, stimulation of osteoclastogenesis (cartilage erosion), osteoporosis); in addition, IL-6 directly induces the synthesis of acute-phase proteins in hepatocytes: CRP, fibrinogen, serum amyloid A protein (SAA, hepcidin, leptin).

In one aspect, the present invention relates to an aqueous pharmaceutical composition of levilimab comprising:

(a) 5-220 mg/ml levilimab;

(b) 0.4-1.8 mg/ml sodium acetate trihydrate;

(c) 20-50 mg/ml polyol and 5-10 mg/ml glycine

or

10-32 mg/ml arginine hydrochloride; and

(d) acetic acid to pH 4.5-6.5.

In some embodiments of the invention, said polyol is selected from mannitol or sorbitol.

In one aspect, the present invention relates to an aqueous pharmaceutical composition of levilimab comprising:

(i) 5-220 mg/ml levilimab;

(ii) 0.4-1.8 mg/ml sodium acetate trihydrate;

(iii) 20-50 mg/ml polyol;

(iv) 5-10 mg/ml glycine; and

(v) acetic acid to pH 4.5-6.5.

The concentration of levilimab contained in the pharmaceutical compositions of the present invention may vary depending on the desired properties of the compositions, as well as on the particular conditions, methods, and purposes of use of the pharmaceutical compositions.

In some embodiments of the invention, said levilimab is present at a concentration of 5-40 mg/ml.

In some embodiments of the invention, said levilimab is present at a concentration of 5 mg/ml.

In some embodiments of the invention, said levilimab is present at a concentration of 5-15 mg/ml.

In some embodiments of the invention, said levilimab is present at a concentration of 10 mg/ml.

In some embodiments of the invention, said levilimab is present at a concentration of 15-25 mg/ml.

In some embodiments of the invention, said levilimab is present at a concentration of 20 mg/ml.

In some embodiments of the invention, said levilimab is present at a concentration of 100-180 mg/ml.

In some embodiments of the invention, said levilimab is present at a concentration of 140-220 mg/ml.

In some embodiments of the invention, said levilimab is present at a concentration of 180-220 mg/ml.

In some embodiments of the invention, said levilimab is present at a concentration of 160-200 mg/ml.

In some embodiments of the invention, said levilimab is present at a concentration of 180 mg/ml.

In some embodiments of the invention, said levilimab is present at a concentration of 200 mg/ml.

In some embodiments of the invention, said sodium acetate trihydrate is present at a concentration of 0.4-1.0 mg/ml.

In some embodiments of the invention, said sodium acetate trihydrate is present at a concentration of 0.4-0.5 mg/ml.

In some embodiments of the invention, said sodium acetate trihydrate is present at a concentration of 0.436 mg/ml.

In some embodiments of the invention, said polyol is present at a concentration of 20-26 mg/ml.

In some embodiments of the invention, said polyol is present at a concentration of 22-24 mg/ml.

In some embodiments of the invention, said polyol is present at a concentration of 23 mg/ml.

In some embodiments of the invention, said polyol may be selected from a sugar alcohol such as mannitol, sorbitol, glycerin, or xylitol, or combinations thereof.

In some embodiments of the invention, said mannitol is present at a concentration of 20-26 mg/ml.

In some embodiments of the invention, said mannitol is present at a concentration of 22-24 mg/ml.

In some embodiments of the invention, said mannitol is present at a concentration of 23 mg/ml.

In some embodiments of the invention, said sorbitol is present at a concentration of 20-26 mg/ml.

In some embodiments of the invention, said sorbitol is present at a concentration of 22-24 mg/ml.

In some embodiments of the invention, said sorbitol is present at a concentration of 23 mg/ml.

In some embodiments of the invention, said combination of mannitol and sorbitol is present at a concentration of 20-26 mg/ml.

In some embodiments of the invention, said combination of mannitol and sorbitol is present at a concentration of 22-24 mg/ml.

In some embodiments of the invention, said combination of mannitol and sorbitol is present at a concentration of 23 mg/ml.

In some embodiments of the invention, said glycine is present at a concentration of 7-8 mg/ml.

In some embodiments of the invention, said glycine is present at a concentration of 7.5 mg/ml.

The desired pH value of the pharmaceutical composition of the present invention may be obtained by adding acetic acid.

In some embodiments of the invention, said acetic acid is added to pH 4.5-5.5.

In some embodiments of the invention, said acetic acid is added to pH 4.5, 5.0, 5.5, 6.0, or 6.5.

In some embodiments of the invention, provided is an aqueous pharmaceutical composition comprising:

(i) 20 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0.

In some embodiments of the invention, provided is an aqueous pharmaceutical composition comprising:

(i) 5 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0.

In some embodiments of the invention, provided is an aqueous pharmaceutical composition comprising:

(i) 10 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0.

In some embodiments of the invention, provided is an aqueous pharmaceutical composition comprising:

(i) 100 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0.

In some embodiments of the invention, provided is an aqueous pharmaceutical composition comprising:

(i) 180 mg/ml levilimab;

(ii) 0.436 mg/ml sodium acetate trihydrate;

(iii) 23 mg/ml mannitol or sorbitol;

(iv) 7.5 mg/ml glycine; and

(v) acetic acid to pH 5.0.

In some embodiments of the invention, provided is an aqueous pharmaceutical composition comprising:

(i) 200 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0.

In some embodiments of the invention, provided is an aqueous pharmaceutical composition comprising:

(i) 220 mg/ml levilimab;

(ii) 0.436 mg/ml sodium acetate trihydrate;

(iii) 23 mg/ml polyol selected from mannitol or sorbitol;

(iv) 7.5 mg/ml glycine; and acetic acid to pH 5.0.

In one aspect, the present invention relates to an aqueous pharmaceutical composition of levilimab comprising:

(i) 5-220 mg/ml levilimab;

(ii) 0.4-1.8 mg/ml sodium acetate trihydrate;

(iii) 10-32 mg/ml arginine hydrochloride; and

(iv) acetic acid to pH 4.5-6.5.

In some embodiments of the invention, said levilimab is present at a concentration of 5-40 mg/ml.

In some embodiments of the invention, said levilimab is present at a concentration of 5 mg/ml.

In some embodiments of the invention, said levilimab is present at a concentration of 5-15 mg/ml.

In some embodiments of the invention, said levilimab is present at a concentration of 10 mg/ml.

In some embodiments of the invention, said levilimab is present at a concentration of 15-25 mg/ml.

In some embodiments of the invention, said levilimab is present at a concentration of 20 mg/ml.

In some embodiments of the invention, said levilimab is present at a concentration of 100-180 mg/ml.

In some embodiments of the invention, said levilimab is present at a concentration of 140-220 mg/ml.

In some embodiments of the invention, said levilimab is present at a concentration of 180-220 mg/ml.

In some embodiments of the invention, said levilimab is present at a concentration of 160-200 mg/ml.

In some embodiments of the invention, said levilimab is present at a concentration of 200 mg/ml.

In some embodiments of the invention, said levilimab is present at a concentration of 180 mg/ml.

In some embodiments of the invention, said sodium acetate trihydrate is present at a concentration of 1.7-1.8 mg/ml.

In some embodiments of the invention, said sodium acetate trihydrate is present at a concentration of 1.744 mg/ml.

In some embodiments of the invention, said arginine hydrochloride is present at a concentration of 18-24 mg/ml.

In some embodiments of the invention, said arginine hydrochloride is present at a concentration of 20-22 mg/ml.

In some embodiments of the invention, said arginine hydrochloride is present at a concentration of 21.1 mg/ml.

In some embodiments of the invention, said acetic acid is added to pH 4.5-5.5.

In some embodiments of the invention, said acetic acid is added to pH 4.5, 5.0, 5.5, 6.0, or 6.5.

In some embodiments of the invention, provided is an aqueous pharmaceutical composition comprising:

(i) 20 mg/ml levilimab;

(ii) 1.744 mg/ml sodium acetate trihydrate;

(iii) 21.1 mg/ml arginine hydrochloride; and

(iv) acetic acid to pH 5.0.

In some embodiments of the invention, provided is an aqueous pharmaceutical composition comprising:

(i) 5 mg/ml levilimab;

(ii) 1.744 mg/ml sodium acetate trihydrate;

(iii) 21.1 mg/ml arginine hydrochloride; and

(iv) acetic acid to pH 5.0.

In some embodiments of the invention, provided is an aqueous pharmaceutical composition comprising:

(i) 10 mg/ml levilimab;

(ii) 1.744 mg/ml sodium acetate trihydrate;

(iii) 21.1 mg/ml arginine hydrochloride; and

(iv) acetic acid to pH 5.0.

In some embodiments of the invention, provided is an aqueous pharmaceutical composition comprising:

(i) 100 mg/ml levilimab;

(ii) 1.744 mg/ml sodium acetate trihydrate;

(iii) 21.1 mg/ml arginine hydrochloride; and

(iv) acetic acid to pH 5.0.

In some embodiments of the invention, provided is an aqueous pharmaceutical composition comprising:

(i) 180 mg/ml levilimab;

(ii) 1.744 mg/ml sodium acetate trihydrate;

(iii) 21.1 mg/ml arginine hydrochloride; and

(iv) acetic acid to pH 5.0.

In some embodiments of the invention, provided is an aqueous pharmaceutical composition comprising:

(i) 200 mg/ml levilimab;

(ii) 1.744 mg/ml sodium acetate trihydrate;

(iii) 21.1 mg/ml arginine hydrochloride; and

(iv) acetic acid to pH 5.0.

In some embodiments of the invention, provided is an aqueous pharmaceutical composition comprising:

(i) 220 mg/ml levilimab;

(ii) 1.744 mg/ml sodium acetate trihydrate;

(iii) 21.1 mg/ml arginine hydrochloride; and

(iv) acetic acid to pH 5.0.

In one aspect, the present invention relates to an aqueous pharmaceutical composition of levilimab comprising:

(i) 162 mg of levilimab;

(ii) 0.392 mg of sodium acetate trihydrate;

(iii) 20.7 mg of mannitol or sorbitol;

(iv) 6.75 mg of glycine;

(v) acetic acid to pH 5.0; and

(vi) water for injections to 0.9 ml.

In one aspect, the present invention relates to an aqueous pharmaceutical composition of levilimab comprising:

(i) 162 mg of levilimab;

(ii) 1.57 mg of sodium acetate trihydrate;

(iii) 18.99 mg of arginine hydrochloride;

(iv) acetic acid to pH 5.0; and

(v) water for injections to 0.9 ml.

In one aspect, the present invention relates to an aqueous pharmaceutical composition of levilimab comprising:

Composition per 0.9 mL:

(i) 162 mg of levilimab;

(ii) 0.392 mg of sodium acetate trihydrate;

(iii) 20.7 mg of mannitol or sorbitol;

(iv) 6.75 mg of glycine;

(v) acetic acid to pH 5.0; and

(vi) water for injections to 0.9 ml.

In one aspect, the present invention relates to an aqueous pharmaceutical composition of levilimab comprising:

Composition per 0.9 mL:

(i) 162 mg of levilimab;

(ii) 1.57 mg of sodium acetate trihydrate;

(iii) 18.99 mg of arginine hydrochloride;

(iv) acetic acid to pH 5.0; and

(v) water for injections to 0.9 ml.

In some embodiments of the invention, said acetic acid is glacial acetic acid.

In some embodiments of the invention, said aqueous pharmaceutical composition of levilimab of the present invention is intended for parenteral administration.

In some embodiments of the invention, said aqueous pharmaceutical composition of levilimab of the present invention is intended for intramuscular, intravenous, or subcutaneous administration.

In some embodiments of the invention, said aqueous pharmaceutical composition of levilimab of the present invention may be administered intravenously as an infusion.

The pharmaceutical compositions of the present invention may be stored in any suitable container. For example, a glass or plastic container, vial, ampoule, syringe, cartridge, autoinjector or bottle of the desired volume.

In some embodiments of the invention, said aqueous pharmaceutical composition is provided in a vial.

In some embodiments of the invention, said vial is a glass or plastic vial.

In some embodiments of the invention, said vial has a volume of 4-20 ml.

In some embodiments of the invention, said vial has a volume of 1 ml, 2 ml, 3 ml, 4 ml, 5 ml, 6 ml, 7 ml, 8 ml, 9 ml, 10 ml, 15 ml or 20 ml.

In some embodiments of the invention, said aqueous pharmaceutical composition is present in a syringe or autoinjector.

In some embodiments of the invention, said syringe or autoinjector is a glass or plastic syringe or autoinjector.

In some embodiments of the invention, said syringe or autoinjector has a capacity of 0.9 ml.

In some embodiments of the invention, said syringe or autoinjector has a capacity of 1 ml.

In some embodiments of the invention, said syringe or autoinjector has a capacity of 2 ml.

In some embodiments of the invention, said syringe or autoinjector may have a volume of 1 ml with a fill volume of 0.9 ml.

In some embodiments of the invention, said aqueous pharmaceutical composition is present in a pre-filled syringe or pre-filled autoinjector.

In some embodiments of the invention, said pre-filled syringe or pre-filled autoinjector is a glass or plastic pre-filled syringe or pre-filled autoinjector.

In some embodiments of the invention, said pre-filled syringe or pre-filled autoinjector has a capacity of 0.9 ml.

In some embodiments of the invention, said pre-filled syringe or pre-filled autoinjector has a capacity of 1 ml.

In some embodiments of the invention, said pre-filled syringe or pre-filled autoinjector has a capacity of 2 ml.

In some embodiments of the invention, said pre-filled syringe or pre-filled autoinjector may have a volume of 1 ml with a fill volume of 0.9 ml.

In one embodiment, the present invention relates to the use of the aqueous pharmaceutical composition of levilimab of the present invention for treating or preventing IL6R-associated diseases or disorders.

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 20-50 mg/ml polyol;

(iv) 5-10 mg/ml glycine; and

(v) acetic acid to pH 4.5-6.5.

for treating or preventing IL6R-associated diseases or disorders.

In one embodiment, the present invention relates to the use of the aqueous pharmaceutical composition of levilimab comprising:

(i) 20 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating or preventing IL6R-associated diseases or disorders.

In one embodiment, the present invention relates to the use of the aqueous pharmaceutical composition of levilimab comprising:

(i) 180 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating or preventing IL6R-associated diseases or disorders.

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 10-32 mg/ml arginine hydrochloride; and

(iv) acetic acid to pH 4.5-6.5

for treating or preventing IL6R-associated diseases or disorders.

In one embodiment, the present invention relates to the use of the aqueous pharmaceutical composition of levilimab comprising:

(i) 20 mg/ml levilimab;

(ii) 1.744 mg/ml sodium acetate trihydrate;

(iii) 21.1 mg/ml arginine hydrochloride; and

(iv) acetic acid to pH 5.0

for treating or preventing IL6R-associated diseases or disorders.

In one embodiment, the present invention relates to the use of the aqueous pharmaceutical composition of levilimab comprising:

(i) 180 mg/ml levilimab;

(ii) 1.744 mg/ml sodium acetate trihydrate;

(iii) 21.1 mg/ml arginine hydrochloride; and

(iv) acetic acid to pH 5.0.

for treating or preventing IL6R-associated diseases or disorders.

In one embodiment, the present invention relates to the use of the aqueous pharmaceutical composition of levilimab comprising:

(i) 162 mg of levilimab;

(ii) 0.392 mg of sodium acetate trihydrate;

(iii) 20.7 mg of polyol selected from mannitol or sorbitol;

(iv) 6.75 mg of glycine;

(v) acetic acid to pH 5.0; and

(vi) water for injection ad 0.9 ml,

for treating or preventing IL6R-associated diseases or disorders.

In one embodiment, the present invention relates to the use of the aqueous pharmaceutical composition of levilimab comprising:

(i) 162 mg of levilimab;

(ii) 1.57 mg of sodium acetate trihydrate;

(iii) 18.99 mg of arginine hydrochloride;

(iv) acetic acid to pH 5.0; and

(v) water for injection ad 0.9 ml,

for treating or preventing IL6R-associated diseases or disorders.

In some embodiments of the invention, said IL6R-associated disease or disorder is selected from: rheumatoid arthritis, juvenile chronic arthritis, scleroderma, graft versus host disease, organ transplant rejection, acute or chronic immune disease associated with organ transplantation, cachexia, adult (acute) respiratory distress syndrome, cytokine release syndrome, Still's disease, systemic scleroderma, Sjogren's syndrome, Takayasu's disease/arteritis, cytokine therapy associated disorders, iridocyclitis, uveitis, optic neuritis, optical neuromyelitis, juvenile rheumatoid arthritis, giant cell arteritis, polyarticular juvenile idiopathic arthritis, systemic-onset juvenile idiopathic arthritis; cancer, in particular multiple myeloma and malignant solid tumors, colorectal cancer, prostate cancer, ovarian cancer.

In some embodiments of the invention, the use of the aqueous pharmaceutical composition of levilimab of the present invention may include administering said composition parenterally.

In some embodiments of the invention, the use of the aqueous pharmaceutical composition of levilimab of the present invention may include administering said composition intramuscularly, intravenously, or subcutaneously.

In some embodiments of the invention, the use of the aqueous pharmaceutical composition of levilimab of the present invention may include administering said composition intravenously as an infusion.

In one embodiment, the present invention relates to the use of the aqueous pharmaceutical composition of levilimab of the present invention for treating rheumatoid arthritis.

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 20-50 mg/ml polyol;

(iv) 5-10 mg/ml glycine; and

(v) acetic acid to pH 4.5-6.5.

for treating rheumatoid arthritis.

In one embodiment, the present invention relates to the use of the aqueous pharmaceutical composition of levilimab comprising:

(i) 20 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating rheumatoid arthritis.

In one embodiment, the present invention relates to the use of the aqueous pharmaceutical composition of levilimab comprising:

(i) 180 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating rheumatoid arthritis.

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 10-32 mg/ml arginine hydrochloride; and

(iv) acetic acid to pH 4.5-6.5

for treating rheumatoid arthritis.

In one embodiment, the present invention relates to the use of the aqueous pharmaceutical composition of levilimab comprising:

(i) 20 mg/ml levilimab;

(ii) 1.744 mg/ml sodium acetate trihydrate;

(iii) 21.1 mg/ml arginine hydrochloride; and

(iv) acetic acid to pH 5.0

for treating rheumatoid arthritis.

In one embodiment, the present invention relates to the use of the aqueous pharmaceutical composition of levilimab comprising:

(i) 180 mg/ml levilimab;

(ii) 1.744 mg/ml sodium acetate trihydrate;

(iii) 21.1 mg/ml arginine hydrochloride; and

(iv) acetic acid to pH 5.0.

for treating rheumatoid arthritis.

In one embodiment, the present invention relates to the use of the aqueous pharmaceutical composition of levilimab comprising:

(i) 162 mg of levilimab;

(ii) 0.392 mg of sodium acetate trihydrate;

(iii) 20.7 mg of polyol selected from mannitol or sorbitol;

(iv) 6.75 mg of glycine;

(v) acetic acid to pH 5.0; and

(vi) water for injection ad 0.9 ml,

for treating rheumatoid arthritis.

In one embodiment, the present invention relates to the use of the aqueous pharmaceutical composition of levilimab comprising:

(i) 162 mg of levilimab;

(ii) 1.57 mg of sodium acetate trihydrate;

(iii) 18.99 mg of arginine hydrochloride;

(iv) acetic acid to pH 5.0; and

(v) water for injection ad 0.9 ml,

for treating rheumatoid arthritis.

In some embodiments of the invention, the use of the aqueous pharmaceutical composition of levilimab of the present invention for treating rheumatoid arthritis may include administering said composition at a dose of levilimab of 162 mg.

In some embodiments of the invention, the use of the aqueous pharmaceutical composition of levilimab of the present invention for treating rheumatoid arthritis may include administering said composition once a week or once every two weeks.

In some embodiments of the invention, the use of the aqueous pharmaceutical composition of levilimab of the present invention for treating rheumatoid arthritis may include administering said composition at a monthly dose of levilimab of 4 mg per kg of body weight.

In some embodiments of the invention, the use of the aqueous pharmaceutical composition of levilimab of the present invention for treating rheumatoid arthritis may include administering said composition at a monthly dose of levilimab of 8 mg per kg of body weight.

In some embodiments of the invention, the use of the aqueous pharmaceutical composition of levilimab of the present invention for treating rheumatoid arthritis may include administering said composition parenterally.

In some embodiments of the invention, the use of the aqueous pharmaceutical composition of levilimab of the present invention for treating rheumatoid arthritis may include administering said composition intramuscularly, intravenously, or subcutaneously.

In some embodiments of the invention, the use of the aqueous pharmaceutical composition of levilimab of the present invention for treating rheumatoid arthritis may include administering said composition intravenously as an infusion.

In some embodiments of the invention, the use of the aqueous pharmaceutical composition of levilimab of the present invention for treating rheumatoid arthritis may further include the use of methotrexate.

In one embodiment, the present invention relates to the use of the aqueous pharmaceutical composition of levilimab of the present invention for treating active rheumatoid arthritis.

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 20-50 mg/ml polyol;

(iv) 5-10 mg/ml glycine; and

(v) acetic acid to pH 4.5-6.5.

for treating active rheumatoid arthritis.

In one embodiment, the present invention relates to the use of the aqueous pharmaceutical composition of levilimab comprising:

(i) 20 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating active rheumatoid arthritis.

In one embodiment, the present invention relates to the use of the aqueous pharmaceutical composition of levilimab comprising:

(i) 180 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating active rheumatoid arthritis.

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 10-32 mg/ml arginine hydrochloride; and

(iv) acetic acid to pH 4.5-6.5

for treating active rheumatoid arthritis.

In one embodiment, the present invention relates to the use of the aqueous pharmaceutical composition of levilimab comprising:

(i) 20 mg/ml levilimab;

(ii) 1.744 mg/ml sodium acetate trihydrate;

(iii) 21.1 mg/ml arginine hydrochloride; and

(iv) acetic acid to pH 5.0

for treating active rheumatoid arthritis.

In one embodiment, the present invention relates to the use of the aqueous pharmaceutical composition of levilimab comprising:

(i) 180 mg/ml levilimab;

(ii) 1.744 mg/ml sodium acetate trihydrate;

(iii) 21.1 mg/ml arginine hydrochloride; and

(iv) acetic acid to pH 5.0.

for treating active rheumatoid arthritis.

In one embodiment, the present invention relates to the use of the aqueous pharmaceutical composition of levilimab comprising:

(i) 162 mg of levilimab;

(ii) 0.392 mg of sodium acetate trihydrate;

(iii) 20.7 mg of polyol selected from mannitol or sorbitol;

(iv) 6.75 mg of glycine;

(v) acetic acid to pH 5.0; and

(vi) water for injection ad 0.9 ml,

for treating active rheumatoid arthritis.

In one embodiment, the present invention relates to the use of the aqueous pharmaceutical composition of levilimab comprising:

(i) 162 mg of levilimab;

(ii) 1.57 mg of sodium acetate trihydrate;

(iii) 18.99 mg of arginine hydrochloride;

(iv) acetic acid to pH 5.0; and

(v) water for injection ad 0.9 ml,

for treating active rheumatoid arthritis.

In some embodiments of the invention, the use of the aqueous pharmaceutical composition of levilimab of the present invention for treating active rheumatoid arthritis may include administering said composition at a dose of levilimab of 324 mg or 648 mg.

In some embodiments of the invention, the use of the aqueous pharmaceutical composition of levilimab of the present invention for treating active rheumatoid arthritis may include administering said composition at a dose of levilimab of 4 mg per kg of body weight.

In some embodiments of the invention, the use of the aqueous pharmaceutical composition of levilimab of the present invention for treating active rheumatoid arthritis may include administering said composition at a dose of levilimab of 8 mg per kg of body weight.

In some embodiments of the invention, the use of the aqueous pharmaceutical composition of levilimab of the present invention for treating active rheumatoid arthritis may include administering said composition once in 2 weeks, or once in 4 weeks, or once in 6 weeks.

In some embodiments of the invention, the use of the aqueous pharmaceutical composition of levilimab of the present invention for treating active rheumatoid arthritis may include administering said composition parenterally.

In some embodiments of the invention, the use of the aqueous pharmaceutical composition of levilimab of the present invention for treating active rheumatoid arthritis may include administering said composition intramuscularly, intravenously, or subcutaneously.

In some embodiments of the invention, the use of the aqueous pharmaceutical composition of levilimab of the present invention for treating active rheumatoid arthritis may include administering said composition intravenously as an infusion.

In some embodiments of the invention, the use of the aqueous pharmaceutical composition of levilimab of the present invention for treating active rheumatoid arthritis may further include the use of methotrexate.

In one embodiment, the present invention relates to the use of the aqueous pharmaceutical composition of levilimab of the present invention for treating or preventing adult (acute) respiratory distress syndrome or cytokine release syndrome

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 20-50 mg/ml polyol;

(iv) 5-10 mg/ml glycine; and

(v) acetic acid to pH 4.5-6.5.

for treating or preventing adult (acute) respiratory distress syndrome or cytokine release syndrome.

In one embodiment, the present invention relates to the use of the aqueous pharmaceutical composition of levilimab comprising:

(i) 20 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating or preventing adult (acute) respiratory distress syndrome or cytokine release syndrome.

In one embodiment, the present invention relates to the use of the aqueous pharmaceutical composition of levilimab comprising:

(i) 180 mg/ml levilimab;

(ii) 0.436 mg/ml sodium acetate trihydrate;

(iii) 23 mg/ml polyol selected from mannitol or sorbitol;

(iv) 7.5 mg/ml glycine; and

(v) acetic acid to pH 5.0

for treating or preventing adult (acute) respiratory distress syndrome or cytokine release syndrome.

In one embodiment, the present invention relates to the use of the aqueous pharmaceutical composition of levilimab comprising:

(i) 5-220 mg/ml levilimab;

(ii) 0.4-1.8 mg/ml sodium acetate trihydrate;

(iii) 10-32 mg/ml arginine hydrochloride; and

(iv) acetic acid to pH 4.5-6.5

for treating or preventing adult (acute) respiratory distress syndrome or cytokine release syndrome.

In one embodiment, the present invention relates to the use of the aqueous pharmaceutical composition of levilimab comprising:

(i) 20 mg/ml levilimab;

(ii) 1.744 mg/ml sodium acetate trihydrate;

(iii) 21.1 mg/ml arginine hydrochloride; and

(iv) acetic acid to pH 5.0

for treating or preventing adult (acute) respiratory distress syndrome or cytokine release syndrome.

In one embodiment, the present invention relates to the use of the aqueous pharmaceutical composition of levilimab comprising:

(i) 180 mg/ml levilimab;

(ii) 1.744 mg/ml sodium acetate trihydrate;

(iii) 21.1 mg/ml arginine hydrochloride; and

(iv) acetic acid to pH 5.0.

for treating or preventing adult (acute) respiratory distress syndrome or cytokine release syndrome.

In one embodiment, the present invention relates to the use of the aqueous pharmaceutical composition of levilimab comprising:

(i) 162 mg of levilimab;

(ii) 0.392 mg of sodium acetate trihydrate;

(iii) 20.7 mg of polyol selected from mannitol or sorbitol;

(iv) 6.75 mg of glycine;

(v) acetic acid to pH 5.0; and

(vi) water for injection ad 0.9 ml,

for treating or preventing adult (acute) respiratory distress syndrome or cytokine release syndrome.

In one embodiment, the present invention relates to the use of the aqueous pharmaceutical composition of levilimab comprising:

(i) 162 mg of levilimab;

(ii) 1.57 mg of sodium acetate trihydrate;

(iii) 18.99 mg of arginine hydrochloride;

(iv) acetic acid to pH 5.0; and

(v) water for injection ad 0.9 ml,

for treating or preventing adult (acute) respiratory distress syndrome or cytokine release syndrome.

In some embodiments of the invention, the use of the aqueous pharmaceutical composition of levilimab of the present invention for treating or preventing adult (acute) respiratory distress syndrome or cytokine release syndrome may include administering said composition at a dose of levilimab of 324 mg or 648 mg.

In some embodiments of the invention, the use of the aqueous pharmaceutical composition of levilimab of the present invention for treating or preventing adult (acute) respiratory distress syndrome or cytokine release syndrome may include administering said composition at a dose of levilimab of 4 mg per kg of body weight.

In some embodiments of the invention, the use of the aqueous pharmaceutical composition of levilimab of the present invention for treating or preventing adult (acute) respiratory distress syndrome or cytokine release syndrome may include administering said composition at a dose of levilimab of 8 mg per kg of body weight.

In some embodiments of the invention, the use of the aqueous pharmaceutical composition of levilimab of the present invention for treating or preventing adult (acute) respiratory distress syndrome or cytokine release syndrome may include administering said composition once, or twice, or three times, or four times at an interval of at least 8 hours.

In some embodiments of the invention, the use of the aqueous pharmaceutical composition of levilimab of the present invention for treating or preventing adult (acute) respiratory distress syndrome or cytokine release syndrome may include administering said composition parenterally.

In some embodiments of the invention, the use of the aqueous pharmaceutical composition of levilimab of the present invention for treating or preventing adult (acute) respiratory distress syndrome or cytokine release syndrome may include administering said composition intramuscularly, intravenously, or subcutaneously.

In some embodiments of the invention, the use of the aqueous pharmaceutical composition of levilimab of the present invention for treating or preventing adult (acute) respiratory distress syndrome or cytokine release syndrome may include administering said composition intravenously as an infusion.

In one embodiment, the present invention relates to a method for treating or preventing an IL6R-associated disease or disorder comprising administering to a subject in need of such prevention or treatment a therapeutically effective amount of the aqueous pharmaceutical composition of levilimab of the present invention.

In some embodiments of the invention, said IL6R-associated disease or disorder is selected from: rheumatoid arthritis, juvenile chronic arthritis, scleroderma, graft versus host disease, organ transplant rejection, acute or chronic immune disease associated with organ transplantation, cachexia, adult (acute) respiratory distress syndrome, Still's disease, systemic scleroderma, Sjogren's syndrome, Takayasu's disease/arteritis, cytokine therapy associated disorders, cytokine release syndrome, iridocyclitis, uveitis, optic neuritis, optical neuromyelitis, juvenile rheumatoid arthritis, giant cell arteritis, polyarticular juvenile idiopathic arthritis, systemic-onset juvenile idiopathic arthritis; cancer, in particular multiple myeloma and malignant solid tumors, colorectal cancer, prostate cancer, ovarian cancer.

In some embodiments of the invention, the method for treating or preventing an IL6R-associated disease or disorder in a subject in need thereof may comprise administering a therapeutically effective amount of the aqueous pharmaceutical composition of levilimab of the present invention parenterally.

In some embodiments of the invention, the method for treating or preventing an IL6R-associated disease or disorder in a subject in need thereof may comprise administering a therapeutically effective amount of the aqueous pharmaceutical composition of levilimab of the present invention intramuscularly, intravenously, or subcutaneously.

In some embodiments of the invention, the method for treating or preventing an IL6R-associated disease or disorder in a subject in need thereof may comprise administering a therapeutically effective amount of the aqueous pharmaceutical composition of levilimab of the present invention intravenously as an infusion.

In one embodiment, the present invention relates to a method for treating rheumatoid arthritis comprising administering to a subject in need of such prevention or treatment a therapeutically effective amount of the aqueous pharmaceutical composition of levilimab of the present invention.

In some embodiments of the invention, the method for treating rheumatoid arthritis in a subject in need thereof may comprise administering the aqueous pharmaceutical composition of levilimab according to the present invention at a dose of levilimab of 162 mg.

In some embodiments of the invention, the method for treating rheumatoid arthritis in a subject in need thereof may comprise administering the aqueous pharmaceutical composition of levilimab according to the present invention once a week or once every two weeks.

In some embodiments of the invention, the method for treating rheumatoid arthritis in a subject in need thereof may comprise administering the aqueous pharmaceutical composition of levilimab according to the present invention at a monthly dose of levilimab of 4 mg per kg of body weight.

In some embodiments of the invention, the method for treating rheumatoid arthritis in a subject in need thereof may comprise administering the aqueous pharmaceutical composition of levilimab according to the present invention at a monthly dose of levilimab of 8 mg per kg of body weight.

In some embodiments of the invention, the method for treating rheumatoid arthritis in a subject in need thereof may comprise administering the aqueous pharmaceutical composition of levilimab according to the present invention parenterally.

In some embodiments of the invention, the method for treating rheumatoid arthritis in a subject in need thereof may comprise administering the aqueous pharmaceutical composition of levilimab according to the present invention intramuscularly, intravenously, or subcutaneously.

In some embodiments of the invention, the method for treating rheumatoid arthritis in a subject in need thereof may comprise administering the aqueous pharmaceutical composition of levilimab according to the present invention intravenously as an infusion.

In some embodiments of the invention, the method for treating rheumatoid arthritis in a subject in need thereof may further comprise administering methotrexate.

In one embodiment, the present invention relates to a method for treating active rheumatoid arthritis comprising administering to a subject in need of such prevention or treatment a therapeutically effective amount of the aqueous pharmaceutical composition of levilimab of the present invention.

In some embodiments of the invention, the method for treating active rheumatoid arthritis in a subject in need thereof may comprise administering the aqueous pharmaceutical composition of levilimab according to the present invention at a dose of levilimab of 324 mg or 648 mg.

In some embodiments of the invention, the method for treating active rheumatoid arthritis in a subject in need thereof may comprise administering the aqueous pharmaceutical composition of levilimab according to the present invention at a dose of levilimab of 4 mg per kg of body weight.

In some embodiments of the invention, the method for treating active rheumatoid arthritis in a subject in need thereof may comprise administering the aqueous pharmaceutical composition of levilimab according to the present invention at a dose of levilimab of 8 mg per kg of body weight.

In some embodiments of the invention, the method for treating active rheumatoid arthritis in a subject in need thereof may comprise administering the aqueous pharmaceutical composition of levilimab according to the present invention once in 2 weeks, or once in 4 weeks, or once in 6 weeks.

In some embodiments of the invention, the method for treating active rheumatoid arthritis in a subject in need thereof may comprise administering the aqueous pharmaceutical composition of levilimab according to the present invention parenterally.

In some embodiments of the invention, the method for treating active rheumatoid arthritis in a subject in need thereof may comprise administering the aqueous pharmaceutical composition of levilimab according to the present invention intramuscularly, intravenously, or subcutaneously.

In some embodiments of the invention, the method for treating active rheumatoid arthritis in a subject in need thereof may comprise administering the aqueous pharmaceutical composition of levilimab according to the present invention intravenously as an infusion.

In some embodiments of the invention, the method for treating active rheumatoid arthritis in a subject in need thereof may further comprise administering methotrexate.

In one embodiment, the present invention relates to a method for treating or preventing adult (acute) respiratory distress syndrome or cytokine release syndrome comprising administering to a subject in need of such prevention or treatment a therapeutically effective amount of the aqueous pharmaceutical composition of levilimab of the present invention.

In some embodiments of the invention, the method for treating or preventing adult (acute) respiratory distress syndrome or cytokine release syndrome in a subject in need thereof may comprise administering the aqueous pharmaceutical composition of levilimab according to the present invention at a dose of levilimab of 324 mg or 648 mg.

In some embodiments of the invention, the method for treating or preventing adult (acute) respiratory distress syndrome or cytokine release syndrome in a subject in need thereof may comprise administering the aqueous pharmaceutical composition of levilimab according to the present invention at a dose of levilimab of 4 mg per kg of body weight.

In some embodiments of the invention, the method for treating or preventing adult (acute) respiratory distress syndrome or cytokine release syndrome in a subject in need thereof may comprise administering the aqueous pharmaceutical composition of levilimab according to the present invention at a dose of levilimab of 8 mg per kg of body weight.

In some embodiments of the invention, the method for treating or preventing adult (acute) respiratory distress syndrome or cytokine release syndrome in a subject in need thereof may comprise administering the aqueous pharmaceutical composition of levilimab according to the present invention once, or twice, or three times, or four times at an interval of at least 8 hours.

In some embodiments of the invention, the method for treating or preventing adult (acute) respiratory distress syndrome or cytokine release syndrome in a subject in need thereof may comprise administering the aqueous pharmaceutical composition of levilimab according to the present invention parenterally.

In some embodiments of the invention, the method for treating or preventing adult (acute) respiratory distress syndrome or cytokine release syndrome in a subject in need thereof may comprise administering the aqueous pharmaceutical composition of levilimab according to the present invention intramuscularly, intravenously, or subcutaneously.

In some embodiments of the invention, the method for treating or preventing adult (acute) respiratory distress syndrome or cytokine release syndrome in a subject in need thereof may comprise administering the aqueous pharmaceutical composition of levilimab according to the present invention intravenously as an infusion.

In one embodiment, the present invention relates to an aqueous pharmaceutical composition of levilimab according to the present invention for use for treating or preventing an IL6R-associated disease or disorder.

In some embodiments of the invention, said IL6R-associated disease or disorder is selected from: rheumatoid arthritis, juvenile chronic arthritis, scleroderma, graft versus host disease, organ transplant rejection, acute or chronic immune disease associated with organ transplantation, cachexia, adult (acute) respiratory distress syndrome, Still's disease, systemic scleroderma, Sjogren's syndrome, Takayasu's disease/arteritis, cytokine therapy associated disorders, cytokine release syndrome, iridocyclitis, uveitis, optic neuritis, optical neuromyelitis, juvenile rheumatoid arthritis, giant cell arteritis, polyarticular juvenile idiopathic arthritis, systemic-onset juvenile idiopathic arthritis; cancer, in particular multiple myeloma and malignant solid tumors, colorectal cancer, prostate cancer, ovarian cancer.

In some embodiments of the invention, the aqueous pharmaceutical composition of levilimab according to the present invention for use for treating or preventing an IL6R-associated disease or disorder may be administered parenterally.

In some embodiments of the invention, the aqueous pharmaceutical composition of levilimab according to the present invention for use for treating or preventing an IL6R-associated disease or disorder may be administered intramuscularly, intravenously, or subcutaneously.

In some embodiments of the invention, the aqueous pharmaceutical composition of levilimab according to the present invention for use for treating or preventing an IL6R-associated disease or disorder may be administered intravenously as an infusion.

In one embodiment, the present invention relates to an aqueous pharmaceutical composition of levilimab according to the present invention for use for treating rheumatoid arthritis.

In some embodiments of the invention, the aqueous pharmaceutical composition of levilimab of the present invention for use for treating rheumatoid arthritis may be administered at a dose of levilimab of 162 mg.

In some embodiments of the invention, the aqueous pharmaceutical composition of levilimab according to the present invention for use for treating rheumatoid arthritis may be administered once a week or once every two weeks.

In some embodiments of the invention, the aqueous pharmaceutical composition of levilimab according to the present invention for use for treating rheumatoid arthritis may be administered at a dose of levilimab of 4 mg per kg of body weight.

In some embodiments of the invention, the aqueous pharmaceutical composition of levilimab according to the present invention for use for treating rheumatoid arthritis may be administered at a dose of levilimab of 8 mg per kg of body weight.

In some embodiments of the invention, the aqueous pharmaceutical composition of levilimab according to the present invention for use for treating rheumatoid arthritis may be administered parenterally.

In some embodiments of the invention, the aqueous pharmaceutical composition of levilimab according to the present invention for use for treating rheumatoid arthritis may be administered intramuscularly, intravenously, or subcutaneously.

In some embodiments of the invention, the aqueous pharmaceutical composition of levilimab according to the present invention for use for treating rheumatoid arthritis may be administered intravenously as an infusion.

In some embodiments of the invention, the aqueous pharmaceutical composition of levilimab according to the present invention for use for treating rheumatoid arthritis may be used in combination with methotrexate.

In one embodiment, the present invention relates to an aqueous pharmaceutical composition of levilimab according to the present invention for use for treating active rheumatoid arthritis.

In some embodiments of the invention, the aqueous pharmaceutical composition of levilimab according to the present invention for use for treating active rheumatoid arthritis may be administered at a dose of levilimab of 324 mg or 648 mg.

In some embodiments of the invention, the aqueous pharmaceutical composition of levilimab according to the present invention for use for treating active rheumatoid arthritis may be administered at a dose of levilimab of 4 mg per kg of body weight.

In some embodiments of the invention, the aqueous pharmaceutical composition of levilimab according to the present invention for use for treating active rheumatoid arthritis may be administered at a dose of levilimab of 8 mg per kg of body weight.

In some embodiments of the invention, the aqueous pharmaceutical composition of levilimab according to the present invention for use for treating active rheumatoid arthritis may be administered once in 2 weeks, or once in 4 weeks, or once in 6 weeks.

In some embodiments of the invention, the aqueous pharmaceutical composition of levilimab according to the present invention for use for treating active rheumatoid arthritis may be administered parenterally.

In some embodiments of the invention, the aqueous pharmaceutical composition of levilimab according to the present invention for use for treating active rheumatoid arthritis may be administered intramuscularly, intravenously, or subcutaneously.

In some embodiments of the invention, the aqueous pharmaceutical composition of levilimab according to the present invention for use for treating active rheumatoid arthritis may be administered intravenously as an infusion.

In some embodiments of the invention, the aqueous pharmaceutical composition of levilimab according to the present invention for use for treating active rheumatoid arthritis may be used in combination with methotrexate.

In one embodiment, the present invention relates to an aqueous pharmaceutical composition of levilimab according to the present invention for use for treating or preventing adult (acute) respiratory distress syndrome or cytokine release syndrome.

In some embodiments of the invention, the aqueous pharmaceutical composition of levilimab according to the present invention for use for treating or preventing adult (acute) respiratory distress syndrome or cytokine release syndrome may be administered at a dose of levilimab of 324 mg or 648 mg.

In some embodiments of the invention, the aqueous pharmaceutical composition of levilimab according to the present invention for use for treating or preventing adult (acute) respiratory distress syndrome or cytokine release syndrome may be administered at a dose of levilimab of 4 mg per kg of body weight.

In some embodiments of the invention, the aqueous pharmaceutical composition of levilimab according to the present invention for use for treating or preventing adult (acute) respiratory distress syndrome or cytokine release syndrome may be administered at a dose of levilimab of 8 mg per kg of body weight.

In some embodiments of the invention, the aqueous pharmaceutical composition of levilimab according to the present invention for use for treating or preventing adult (acute) respiratory distress syndrome or cytokine release syndrome may be administered once, or twice, or three times, or four times at an interval of at least 8 hours.

In some embodiments of the invention, the aqueous pharmaceutical composition of levilimab according to the present invention for use for treating or preventing adult (acute) respiratory distress syndrome or cytokine release syndrome may be administered parenterally.

In some embodiments of the invention, the aqueous pharmaceutical composition of levilimab according to the present invention for use for treating or preventing adult (acute) respiratory distress syndrome or cytokine release syndrome may be administered intramuscularly, intravenously, or subcutaneously.

In some embodiments of the invention, the aqueous pharmaceutical composition of levilimab according to the present invention for use for treating or preventing adult (acute) respiratory distress syndrome or cytokine release syndrome may be administered intravenously as an infusion.

In one aspect, the present invention relates to a method for producing an aqueous pharmaceutical composition of levilimab comprising combining 5-220 mg/ml levilimab and

0.4-1.8 mg/ml sodium acetate trihydrate;

20-50 mg/ml polyol;

5-10 mg/ml glycine; and

acetic acid to pH 4.5-6.5.

In one aspect, the present invention relates to a method for producing an aqueous pharmaceutical composition of levilimab comprising combining 5-220 mg/ml levilimab and

0.4-1.8 mg/ml sodium acetate trihydrate;

10-32 mg/ml arginine hydrochloride; and

acetic acid to pH 4.5-6.5.

In one aspect, the present invention relates to a method for producing an aqueous pharmaceutical composition of levilimab, wherein said acetic acid is glacial acetic acid.

The present invention relates to suitable aqueous pharmaceutical compositions of the anti-IL-6R antibody levilimab. One aqueous pharmaceutical composition may comprise levilimab, an acetate-based buffer, polyol, glycine, and acetic acid. Another aqueous pharmaceutical composition may contain levilimab, an acetate-based buffer, arginine hydrochloride, and acetic acid.

The acetate-based buffer may be the result of combining acetic acid with sodium acetate trihydrate. It will be understood that even though sodium acetate trihydrate may be used as a salt for the acetate-based buffer, any other acetate salt, such as potassium acetate, may be used for the acetate-based buffer without departing from the teachings of the present invention.

In the aqueous pharmaceutical compositions of levilimab according to the present invention, arginine, in particular, L-arginine, or arginine hydrochloride may be used.

The present invention relates to the use of the aqueous pharmaceutical composition of levilimab according to the present invention for treating or preventing an IL6R-associated disease or disorder.

Diseases or disorders that may be treated with the compositions provided herein consist of, without limitation, rheumatoid arthritis, juvenile chronic arthritis, scleroderma, graft versus host disease, organ transplant rejection, acute or chronic immune disease associated with organ transplantation, cachexia, adult (acute) respiratory distress syndrome, Still's disease, systemic scleroderma, Sjogren's syndrome, Takayasu's disease/arteritis, cytokine therapy associated disorders, cytokine release syndrome, iridocyclitis, uveitis, optic neuritis, optical neuromyelitis, juvenile rheumatoid arthritis, giant cell arteritis, polyarticular juvenile idiopathic arthritis, systemic-onset juvenile idiopathic arthritis; cancer, in particular multiple myeloma and malignant solid tumors, colorectal cancer, prostate cancer, ovarian cancer.

The pharmaceutical compositions provided may be administered to a subject in need of treatment by systemic injection, for example, by intravenous or subcutaneous injection, or by intramuscular injection; or by direct injection.

The aqueous pharmaceutical composition of levilimab according to the present invention may be used after dilution. To this end, the required volume of the composition is transferred from a vial to an infusion container comprising a sterile 0.9% sodium chloride solution or a sterile 5% dextrose solution. The resulting solution is stirred by gently turning the infusion container over to avoid foaming.

In one embodiment of the invention, a dose may be delivered as one or more than one infusion. The dose may be delivered as one, two or three infusions. In some embodiments of the invention, the duration of treatment may be from one or several infusions.

The therapeutically effective amount of aqueous compositions comprising levilimab according to the present invention in the provided formulations depends on the condition to be treated, the severity of the condition, the previous therapy and the patient's history and response to the therapeutic agent. A suitable dose can be adjusted by the decision of the attending physician so that it can be administered to the patient once or through several injections.

In one embodiment, the effective amount of levilimab per dose for a patient is about 4 mg per kg of body weight or 8 mg per kilogram of body weight.

The dose may be delivered as one or more than one injection. The dose may be delivered as one, two or three injections. A single injection may contain 0.9 ml, 1 ml, 1.8 ml, or 2 ml of the composition disclosed herein.

In one embodiment, the aqueous pharmaceutical composition of levilimab of the present invention for use for treating rheumatoid arthritis may be administered at a dose of levilimab of 162 mg by a single injection.

In one embodiment, the aqueous pharmaceutical composition of levilimab of the present invention for use for treating active rheumatoid arthritis may be administered at a dose of 324 mg by a single injection.

In one embodiment, the aqueous pharmaceutical composition of levilimab of the present invention for use for treating active rheumatoid arthritis may be administered at a dose of 324 mg by two injections of 162 mg each.

In one embodiment, the aqueous pharmaceutical composition of levilimab of the present invention for use for treating active rheumatoid arthritis may be administered at a dose of 648 mg by a single injection.

In one embodiment, the aqueous pharmaceutical composition of levilimab of the present invention for use for treating active rheumatoid arthritis may be administered at a dose of 648 mg by two injections of 324 mg each.

In one embodiment, the aqueous pharmaceutical composition of levilimab of the present invention for use for treating active rheumatoid arthritis may be administered at a dose of 648 mg by four injections of 162 mg each.

In one embodiment, the aqueous pharmaceutical composition of levilimab according to the present invention for use for treating or preventing adult (acute) respiratory distress syndrome or cytokine release syndrome may be administered at a dose of 324 mg by a single injection.

In one embodiment, the aqueous pharmaceutical composition of levilimab according to the present invention for use for treating or preventing adult (acute) respiratory distress syndrome or cytokine release syndrome may be administered at a dose of 324 mg by two injections of 162 mg each.

In one embodiment, the aqueous pharmaceutical composition of levilimab according to the present invention for use for treating or preventing adult (acute) respiratory distress syndrome or cytokine release syndrome may be administered at a dose of 648 mg by a single injection.

In one embodiment, the aqueous pharmaceutical composition of levilimab according to the present invention for use for treating or preventing adult (acute) respiratory distress syndrome or cytokine release syndrome may be administered at a dose of 648 mg by two injections of 324 mg each.

In one embodiment, the aqueous pharmaceutical composition of levilimab according to the present invention for use for treating or preventing adult (acute) respiratory distress syndrome or cytokine release syndrome may be administered at a dose of 648 mg by four injections of 162 mg each.

In another embodiment, the pharmaceutical compositions of the present invention may be prepared as a bulk formulation, and in essence, the components of the pharmaceutical composition are present in amounts higher than may be required for administration and are diluted accordingly before administration.

Alternatively, a pharmaceutical composition may be frozen, spray-dried or lyophilized and reconstituted before application in an appropriate sterile carrier. Lyophilisation can be performed using techniques known in the art which include various steps, such as freezing, annealing, primary and secondary drying.

The pharmaceutical compositions may be administered as a single therapeutic agent or in combination with additional therapeutic agents as needed. Thus, in one embodiment, the provided methods for treatment and/or prevention are used in combination with administration of a therapeutically effective amount of another active agent. The other active agent may be administered before, during or following the administration of the pharmaceutical compositions of the present invention. The other active agent may be administered as part of the provided composition or, alternatively, as a separate formulation.

The pharmaceutical compositions, if desired, may be provided in a vial, package, or in a dispenser device, which may contain one or more unit dosage forms containing the active ingredient. In one embodiment, the dispenser device may comprise a syringe containing a single dose of the liquid formulation ready for injection. The syringe may be accompanied by instructions for administration.

In another embodiment, the present invention relates to a kit or container containing the aqueous pharmaceutical composition according to the invention. The kit may also be accompanied by instructions for use.

Methods

1. Preparation of Levilimab Samples.

Antibody samples at a concentration of 5-20 mg/ml were prepared in Stirred Cell (Millipore) under pressure. To this end, the initial antibody formulation was placed in a cell, the protein was concentrated under a compressed air stream to a concentration of 10 mg/ml under continuous stirring, at least 10-fold volume of the aqueous solution with the target formulation comprising buffering agents, osmotic agents and, if necessary, additional water soluble stabilizers was then added to the cell. After diafiltration the antibody was concentrated to a concentration of about 30 mg/ml, unloaded from the cell, and the exact protein concentration was measured by UV spectroscopy. An appropriate solution of excipients was then added to the sample to prepare a solution with the target concentration of protein.

Protein samples at 20 mg/ml or higher were prepared in Pellicon cassettes (Millipore) in a tangential flow mode. To this end, the initial antibody formulation was placed in a diafiltration tank, the protein was concentrated to a concentration of about 45-50 mg/ml, at least 10-fold volume of the solution with the target formulation comprising buffering agents, and, if necessary, additional water-soluble stabilizers was then supplied to the system. After diafiltration the antibody was concentrated to a concentration of 100 mg/ml, unloaded from the system, osmotic agents and stabilizers were added, the concentrating was continued to a concentration exceeding the target one, unloaded from the system, and the exact protein concentration was determined. An appropriate solution of excipients was then added to the sample to prepare a solution with the target concentration of protein.

When obtaining formulations comprising solubilizers, the surfactant concentrates were added to the antibody following diafiltering and concentrating with the final dilution of the antibody to the target concentration with a solution of excipients.

During aseptic filling into the final container (for example, a sterile glass/plastic vessel, vial or syringe), the antibody solution was filtered using a 0.22 μm sterilizing membrane.

2. Determination of Protein Concentration in Test Samples

The protein concentration was measured by UV spectroscopy at a wavelength of 280 nm in UV transparent plates.

Each sample was diluted with an appropriate solution of excipients to a concentration of ˜0.5 mg/ml. 150 μl of the diluted sample was placed to UV spectroscopy plate well. Optical density of solutions in the plate wells was measured using a plate spectrophotometer at a wavelength of 280 nm. An appropriate solution of excipients was used as a reference solution.

Concentration (mg/ml) of protein (C) was calculated using the following formula:

$$C = \frac{A ( 280 ) * b}{ε * l} ,$$

where

A₂₈₀ is a value of optical density at a wavelength of 280 nm;

ε is an extinction coefficient of test protein;

b is a total dilution factor for a sample;

l is layer thickness in a plate well; for 150 μl, l=0.42 cm.

3. Determination of Protein Aggregation Temperature by Dynamic Light Scattering.

The point of aggregation of the test proteins (at a concentration of 1 mg/ml) was determined using the Zetasizer Nano ZSP instrument. To this end, 0.5 ml of the solution was placed in a quartz dust-free cuvette that was gradually heated in the device under constant measurement of the scattered light intensity.
Analytical model: Protein analysis.
Mode: Temperature trend, mod: Protein aggregation point. 50 to 83° C. at a heating increment of 1.5° C.
Keeping for 30 seconds at temperature before starting the measurement.
The scattered light intensity was detected at an angle of θ=173°.
At each point, average value from 13 measurements in 1 replication.

The temperature trend and aggregation point were determined using the instrument software.

4. Determination of Colloidal Stability by PEG Aggregation

A solution of PEG 6000 with a mass concentration of 20-25% in the test excipient composition was prepared. The resulting solutions were filtered through a 0.45 μm Durapore filter.

An estimated amount of the sample, excipient solution, and 20-25% PEG 6000 solution were transferred to 96 well UV plates so that the concentration of PEG 6000 in a number of wells ranged from 0 to 18% and protein concentration in each well was 1 mg/ml. All solutions prepared in wells were thoroughly mixed by pipetting.

Turbidity of solutions was then evaluated visually, and optical density of solutions at a wavelength of 400 nm was measured.

Protein precipitation in the presence of PEG is associated with the effect of volume substitution, i.e., protein is sterically excluded from regions of solvent by the polymer (L. Li, A. Kantor, N. Warne. Application of a PEG precipitation method for solubility screening: A tool for developing high protein concentration formulations, Protein Sci. 2013 August; 22(8): 1118-1123). This results in protein concentration until its solubility is exceeded and it is precipitated. The less stable is a sample, the lower is PEG 6000 concentration at which the sample will form visible aggregates (opalescence).

5. Determination of Thermal Stability Under 50° C. Thermal Stress

Test samples were divided into 2 aliquots of 150 μl each and placed into separate glass vials: 1 vial per composition was stored in a refrigerator at 2-8° C., the rest vials were placed in a thermostat and incubated at a required temperature for the specified period of time. When selecting control points or after heating, the vials were removed from the thermostat, kept at room temperature for about 15 minutes, and transferred for analysis.

6. Determination of Colloidal Stability During Shaking.

Test samples were divided into 2 aliquots of 150 μl each and placed into glass vials, 1 vial per formulation was stored in a refrigerator at 5±3° C., the rest vials were placed into a thermal shaker and shaken at a speed of 800 rpm at 5±3° C. for the specified period. During the selection of control points or after the stress, the vials were removed from the thermal shaker and transferred for analysis.

7. Determination of Colloidal Stability During Freezing and Thawing.

Test samples were divided into 2 aliquots and placed into plastic vials: 1 vial per formulation was stored in a refrigerator at 5±3° C., the rest vials were stored in a freezer at minus 16-20° C. for the specified period of time. After the stress, the vials were removed from the freezer, kept at room temperature until the content was completely thawed; the solutions were mixed using a vortex and transferred for analysis.

8. Accelerated Storage.

The test samples at a protein concentration of 20, 180, and 220 mg/ml were divided into separate aliquots (one for the input control—it is allowed to transfer for analysis once for all studies at the start of storage) and placed in separate sterile glass vials and syringes: a portion of the vials and syringes for each composition was placed in the refrigerator for storage at 5±3° C. (input control), the rest were placed in a thermostat and incubated at 25° C. for 6 months, periodically selecting control points according to the plan. When selecting control points and after storing, the vials and syringes were removed from the thermostat and transferred for analysis.

9. Determination of Sample Purity by Size-Exclusion High-Performance Liquid Chromatography (SE HPLC).
Tosoh column TSK-GelG3000SWXL 7.8 mm ID×30 cm, cat. No 08541.
Column temperature: 25.
Mobile phase flow rate: 0.7 ml/min.
Injection volume: 10 μl.
Sample concentration: 5 mg/ml.
Detector wavelength: 220 and 280 nm.
Elution time: 23 min.
Mobile phase: Disodium hydrogen phosphate anhydrous 7.1 mg/ml. Sodium chloride 17.54 mg/ml.

The mobile phase pH was adjusted to 7.0 with orthophosphoric acid.

10. Determination of Charged Form Profile by Ion Exchange High Performance Liquid Chromatography (IE HPLC).
Column: TSKgel CM-STAT, 4.6 mm×100 mm, 7 micron particle size (Tosoh Bioscience LLC, Japan, 21966)
Eluent A: Solution of 10 mM disodium hydrogen phosphate anhydrous, pH=6.8
Fluent B: Solution of 10 mM disodium hydrogen phosphate anhydrous, 200 mM NaCl, pH=6.8
Flow rate: 0.7 ml/min.
Column temperature: 35° C.
Temperature of autosampler: 5° C.
Detector: UV, 280 nm
Reference wavelength: 360 nm, 100 nm bandwidth
Sample volume: 40 μl
Elution mode: Fluent A 100→0→100%
Eluent B 0→100→0%
Chromatography time: 60 min.

The test sample was diluted to a concentration of 1.0 mg/ml and treated with carboxypeptidase B (1% of the sample volume) for 2 hours at a temperature of (37±1) ° C.

11. Determination of Homogeneity by Vertical Polyacrylamide Gel Electrophoresis Under Reducing and Non-Reducing Conditions (Red. VPAGE and Non-Red. VPAGE).

PAAG was prepared in glass plates in the presence of sodium dodecyl sulfate, said plates consisting of a concentrating layer of 4% PAAG and a separating layer of 12.5% PAAG (under reducing conditions)/8% PAAG (under non-reducing conditions).

An electrophoresis chamber was assembled and installed in accordance with a vertical electrophoresis apparatus user manual. Probes were prepared by diluting samples with purified water to a final concentration of 1 mg/ml. A volume equivalent of 40 μg was taken, and the prepared probes of the test sample were mixed in a ratio of 3:1 (volume/volume) with a 4× sample buffer solution containing 2-mercaptoethanol (reducing conditions) and not containing 2-mercaptoethanol (non-reducing conditions) and stirred. The resulting solutions were incubated at (99±1) ° C. for 3 min (samples containing 2-mercaptoethanol) and at (99±1) ° C. for 1 min (samples without 2-mercaptoethanol). The solutions were cooled to room temperature, mixed, and transferred to PAAG wells under an electrode buffer solution layer.

Electrophoresis was performed in constant current mode using a water-cooling system. Parameters of power supply were set: the voltage was 110 V during passing of the dye front through the concentrating gel. After moving of the dye front into the lower separation gel at the level of 5-7 mm, the voltage was increased to 180 V. The power supply was turned off when the dye front reached the bottom line of the gel.

After electrophoresis, the gels were detached from the glasses, and the proteins were fixed in a fixing solution for 16-18 hours at room temperature. The gels were then stained (in an Acid Blue 83 solution) and washed to obtain a clear visualization of the bands. The gels were scanned. The purity and impurities in the test samples were evaluated using GelPro software.

12. Determination of Relative Specific Activity.

Specific activity was determined using an antiproliferative test on DS-1 cell culture. Samples were processed using the TecanEvo 200 robotic platform; RPMI1640 comprising 2 mM Gln, 10% FBS, 1 mM sodium pyruvate, and 50 μg/ml gentamicin was used as an assay medium (medium for quantitative determination).

The test antibody sample was diluted using the assay medium to a concentration of 5 mg/ml and placed into the robotic platform. TecanEvo 200 was used to prepare three independent dilutions of the standard and test sample at concentrations of 1 000 000, 250 000, 100 000, 25 000, 5000, 2500, 1000, 250, 50, 5.0 ng/ml using the assay medium. The dilutions and assay medium were transferred to culture plates, and a DS-1 cell suspension at a concentration of (1.5±0.1)×10⁵ cells/ml and IL6 working solution, 7.5 ng/ml, were added to the dilutions of the test and standard samples. Culture plates were placed in a CO₂ incubator, incubated at a temperature of (37±1) ° C. in humidified air with a carbon dioxide content of 5% for 70-72 hours.

After the incubation period, Alamar blue dye was added to the wells of the culture plate and the plates were incubated under the same conditions until the gradient color developed. The fluorescence intensity was measured at excitation/emission wavelength of 544/590 nm. Using the Magellan ver 7.2 software, we plotted the graph of dependence of fluorescence intensity on protein concentration. The relative specific activity of the test samples was determined as the ratio of ED50 of the standard sample to that of the test sample, expressed as a percentage.

13. Processing of Results.

The absolute change in quality indicators when under stresses was calculated by the formula:

Δ=(after-stress value−before-stress value)

Absolute change in the charged form profile was calculated by the formula:

Δ=|acidic fraction content before stress−acidic fraction content after stress|+|alkaline fraction content before stress−alkaline fraction content after stress|+|dominant fraction content before stress−dominant fraction content after stress|

### EXAMPLES

### Example 1. Selection of Buffer System

In this study, 2 typical buffer systems, acetate, and histidine buffer systems, that are suitable for parenteral administration were selected as the basis of the pharmaceutical composition.

To assess the suitability of the buffer systems in relation to the processing characteristics of the pharmaceutical composition, the effect of the nature of the buffer solution on the colloidal stability of the protein during concentration thereof was studied. As a response, the sample filtration time through a 0.22-micron sterilizing filter was measured. The test pharmaceutical compositions are shown in Table 1.

TABLE 1
Test formulations

| | |
|---|---|
| 5 Acet buf | Levilimab from 100 to 180 mg/ml |
| | Sodium acetate trihydrate 0.436 mg/ml |
| | Acetic acid to pH 5.0 |
| | Water for injections to 1 ml |
| 5 His buf | Levilimab from 100 to 180 mg/ml |
| | Histidine 0.23 mg/ml |
| | Histidine hydrochloride monohydrate 0.74 mg |
| | Water for injections to 1 ml |

Measurement of Filtration Time.

The samples were concentrated according to method 1. Upon reaching a concentration of 100 mg/ml, 130 mg/ml and 180 mg/ml, the time of sterilizing filtration of the pharmaceutical composition was measured. The results of the study of the time of sterilizing filtration are shown in Table 2.

TABLE 2
Time of sterilizing filtration

| | Centrifuge | | | | Final |
|---|---|---|---|---|---|
| | rotor | Filtration | Filtration | Filtration | volume of |
| | rotational | time (100 | time (130 | time (180 | protein |
| Formulation | speed, | mg/ml), | mg/ml), | mg/ml), | solution, |
| name | rpm | min | min | min | ml |
| 5 Acet buf | 7500 → | 10 | 20 | 35 | 0.245 |
| | 10000 | | | | |
| 5 His buf | 7500 → | 10 | 30 | 60 | 0.245 |
| | 10000 | | | | |

The use of the acetate buffer system reduces the filtration time of the pharmaceutical composition containing 180 mg/ml protein by approximately 1.7 times as compared to the histidine buffer system, which indicates better solubility and colloidal stability thereof.

### Example 2. Initial Selection of Osmotic Agent

Test Formulations

Excipients suitable for parenteral administration were studied to be used as osmotic agents. The test formulations are shown in Table 3.

TABLE 3
Test formulations

| 5 Acet. Buf. + | Levilimab | 5 | mg/ml |
|---|---|---|---|
| Tre | Sodium acetate trihydrate | 0.436 | mg |
| | Trehalose dihydrate | 100 | mg |
| | Acetic acid glacial | to pH 5 | |
| | Water for injections | to 1 ml | |
| 5 Acet. Buf. + | Levilimab | 5 | mg/ml |
| Mann | Sodium acetate trihydrate | 0.436 | mg |
| | Mannitol | 45 | mg |
| | Acetic acid glacial | to pH 5 | |
| | Water for injections | to 1 ml | |
| 5 Acet. Buf. + | Levilimab | 5 | mg/ml |
| 300 Gly | Sodium acetate trihydrate | 0.436 | mg |
| | Glycine | 23 | mg |
| | Acetic acid glacial | to pH 5 | |
| | Water for injections | to 1 ml | |
| 5 Acet. Buf. + | Levilimab | 5 | mg/ml |
| 200 Arg | Sodium acetate trihydrate | 0.436 | mg |
| | Arginine hydrochloride | 42.1 | mg |
| | Acetic acid glacial | to pH 5 | |
| | Water for injections | to 1 ml | |
| 5 Acet. Buf. + | Levilimab | 5 | mg/ml |
| 100 Arg + | Sodium acetate trihydrate | 0.436 | mg |
| Mann | Arginine hydrochloride | 21.1 | mg |
| | Mannitol | 45 | mg |

| | |
|---|---|
| Acetic acid glacial | to pH 5 |
| Water for injections | to 1 ml |

Determination of Colloidal Stability by PEG Aggregation

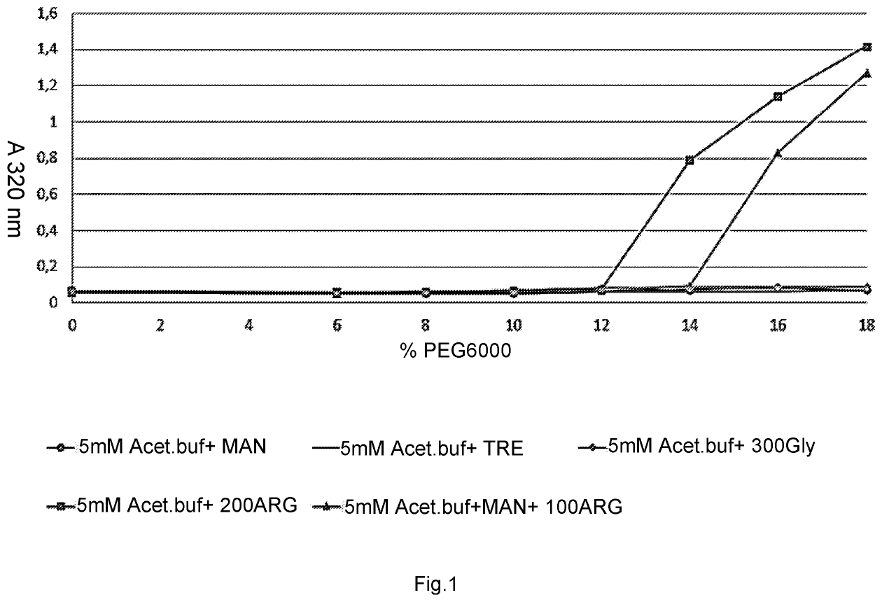

The PEG aggregation assay enables to simulate the direct concentration of levilimab by displacing same by the inert polymer PEG 6000, as well as to comparatively assess the theoretical solubility of the antibody in various formulations. The study was performed according to method 4. Data on the average optical density of solutions is shown in Table 4. The results are also shown in FIG. 1.

Determination of Thermal Stability.

Thermal stability was measured using methods 3 and 5. Before and following thermal stress, total impurity content was measured by the SE HPLC method using method 9.

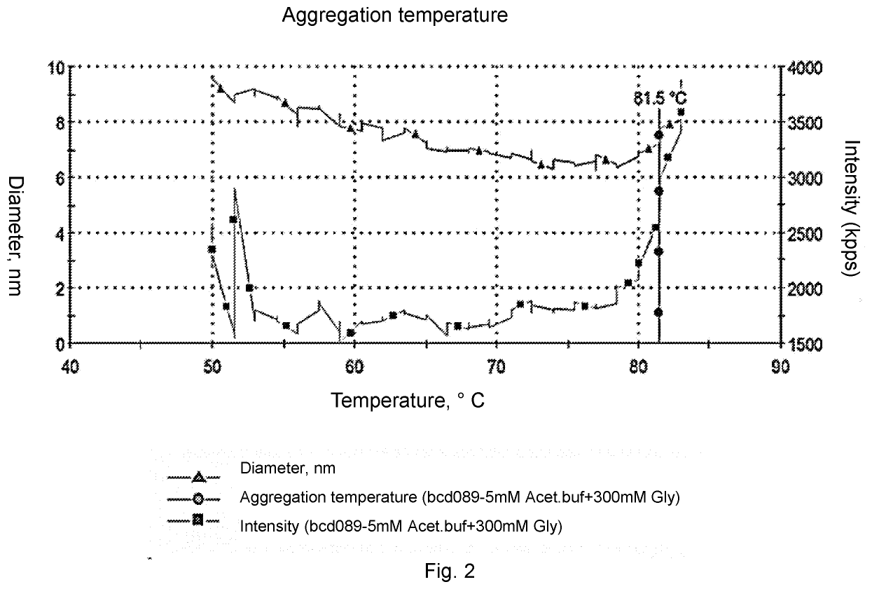

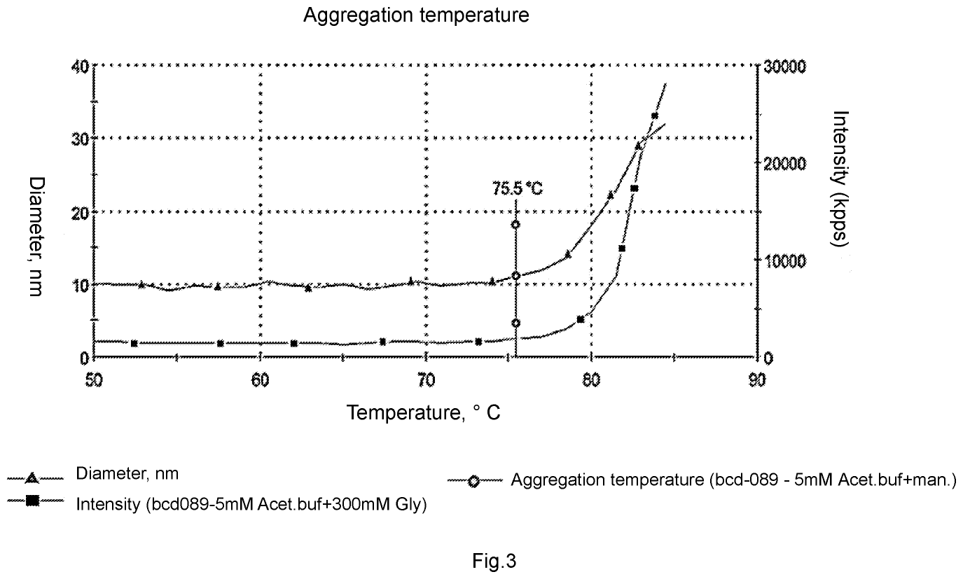

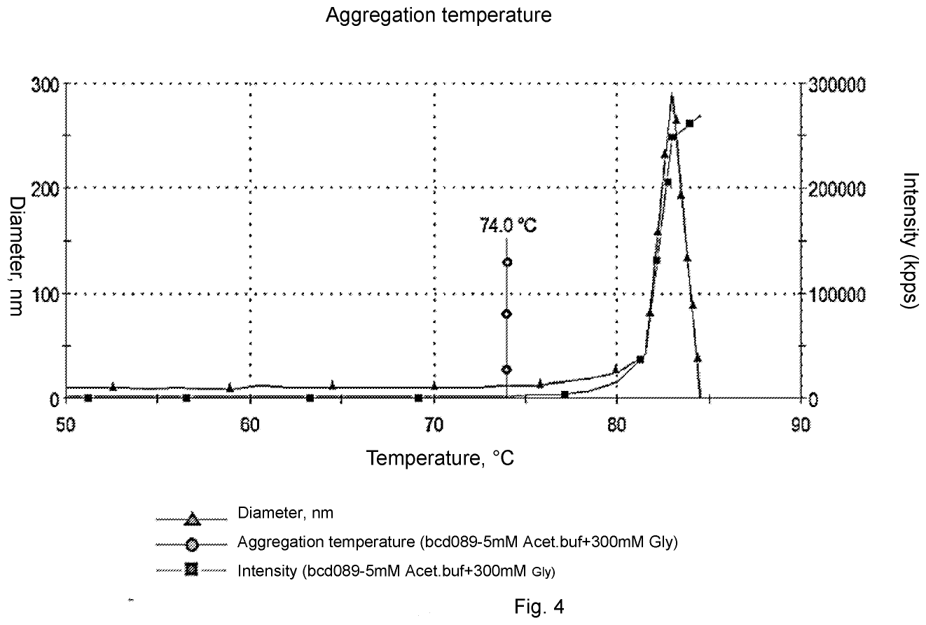

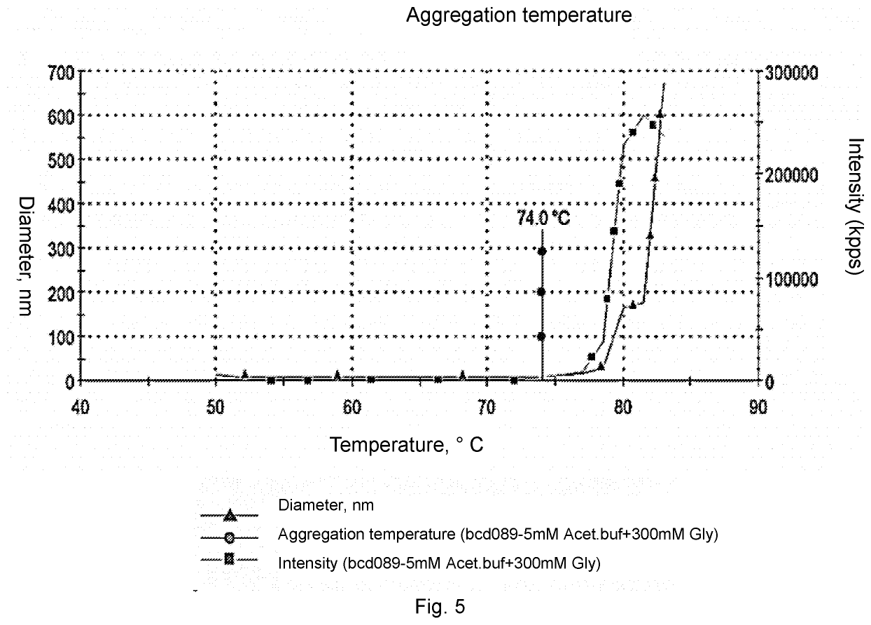

The results are shown in Table 5 and in FIGS. 2, 3, 4 and 5.

TABLE 5
Results of determination of thermal stability

| | Impurity | Increase in impurity | Aggregation |
|---|---|---|---|
| | content, | content following 96 | temperature, |
| Formulation name | % | h thermal stress, % | ° C. |
| Acet | 0.27 | 4.69 | — |
| 5 Acet. Buf. + Mann | 0.22 | 4.61 | 75.5 |
| 5 Acet. Buf. + Tre | 0.21 | 5.39 | — |
| 5 Acet. Buf. + 300 Gly | 0.21 | 4.27 | 81.5 |
| 5 Acet. Buf. + 200 Arg | 0.23 | 5.23 | 74 |
| 5 Acet. Buf. + 100 | 0.23 | 3.75 | 74 |
| Arg + Mann | | | |

Pharmaceutical compositions based on the acetate buffer system comprising mannitol, trehalose dihydrate, and glycine as osmotic agents demonstrated better colloidal stability during PEG aggregation.

High thermal stability was demonstrated by compositions based on the acetate buffer solution comprising glycine and mannitol as osmotic agents.

### Example 3. Screening of Osmotic Agents and Stabilizers

Excipients suitable for parenteral administration were used to screen osmotic agents and stabilizers. The test formulations are shown in Table 6. Pharmaceutical compositions comprising levilimab at a concentration of 10 mg/ml in the test formulations were prepared according to technique 2.

TABLE 6
Test formulations

| Ac + SORB | Levilimab | 10 | mg/ml |
|---|---|---|---|
| | Sodium acetate trihydrate | 1.742 | mg/ml |
| | Acetic acid glacial | to pH 5.0 | |
| | Sorbitol | 45 | mg/ml |
| Ac + SORB + PLX188 | Levilimab | 10 | mg/ml |
| | Sodium acetate trihydrate | 1.742 | mg/ml |
| | Acetic acid glacial | to pH 5.0 | |
| | Sorbitol | 45 | mg/ml |
| | Poloxamer 188 | 1 | mg/ml |
| Ac + SORB + Ser | Levilimab | 10 | mg/ml |
| | Sodium acetate trihydrate | 1.742 | mg/ml |
| | Acetic acid glacial | to pH 5.0 | |
| | Sorbitol | 45 | mg/ml |
| | L-Serine | 20 | mM |
| Ac + SORB + Gly | Levilimab | 10 | mg/ml |
| | Sodium acetate trihydrate | 1.742 | mg/ml |
| | Acetic acid glacial | to pH 5.0 | |
| | Sorbitol | 45 | mg/ml |
| | Glycine | 100 | mM |
| Ac + SORB + Arg + | Levilimab | 10 | mg/ml |
| Glu | Sodium acetate trihydrate | 1.742 | mg/ml |
| | Acetic acid glacial | to pH 5.0 | |
| | Sorbitol | 45 | mg/ml |
| | L-arginine hydrochloride | 50 | mM |
| | monohydrate | | |
| | Sodium glutamate monohydrate | 50 | mM |
| Ac + SORB + Met | Levilimab | 10 | mg/ml |
| | Sodium acetate trihydrate | 1.742 | mg/ml |
| | Acetic acid glacial | to pH 5.0 | |
| | Sorbitol | 45 | mg/ml |
| | L-methionine | 1 | mM |
| Ac + SORB + 20Met | Levilimab | 10 | mg/ml |
| | Sodium acetate trihydrate | 1.742 | mg/ml |
| | Acetic acid glacial | to pH 5.0 | |
| | Sorbitol | 45 | mg/ml |
| | L-methionine | 100 | mM |
| Ac + MANN | Levilimab | 10 | mg/ml |
| | Sodium acetate trihydrate | 1.742 | mg/ml |
| | Acetic acid glacial | to pH 5.0 | |
| | Mannitol | 45 | mg/ml |
| Ac + MANN + | Levilimab | 10 | mg/ml |
| PLX188 | Sodium acetate trihydrate | 1.742 | mg/ml |
| | Acetic acid glacial | to pH 5.0 | |
| | Mannitol | 45 | mg/ml |
| | Poloxamer 188 | 1 | mg/ml |
| AC + MANN + | Levilimab | 10 | mg/ml |
| 10SBECD | Sodium acetate trihydrate | 1.742 | mg/ml |
| | Acetic acid glacial | to pH 5.0 | |
| | Mannitol | 45 | mg/ml |
| | Cyclodextrin sulfobutyl ether | 10 | mg/ml |
| Ac + MANN + | Levilimab | 10 | mg/ml |
| 30SBECD | Sodium acetate trihydrate | 1.742 | mg/ml |
| | Acetic acid glacial | to pH 5.0 | |
| | Mannitol | 45 | mg/ml |
| | Cyclodextrin sulfobutyl ether | 30 | mg/ml |
| Ac + MANN + | Levilimab | 10 | mg/ml |
| 10HPBCD | Sodium acetate trihydrate | 1.742 | mg/ml |
| | Acetic acid glacial | to pH 5.0 | |
| | Mannitol | 45 | mg/ml |
| | Hydroxypropyl-β-cyclodextrin | 10 | mg/ml |
| Ac + MANN + | Levilimab | 10 | mg/ml |
| 30HPBCD | Sodium acetate trihydrate | 1.742 | mg/ml |
| | Acetic acid glacial | to pH 5.0 | |
| | Mannitol | 45 | mg/ml |
| | Hydroxypropyl-β-cyclodextrin | 30 | mg/ml |
| Ac + MANN + Lys | Levilimab | 10 | mg/ml |
| | Sodium acetate trihydrate | 1.742 | mg/ml |
| | Acetic acid glacial | to pH 5.0 | |
| | Mannitol | 45 | mg/ml |
| | L-lysine | 20 | mM |
| Ac + MANN + Ser | Levilimab | 10 | mg/ml |
| | Sodium acetate trihydrate | 1.742 | mg/ml |
| | Acetic acid glacial | to pH 5.0 | |
| | Mannitol | 45 | mg/ml |
| | L-Serine | 20 | mM |
| Ac + MANN + 20Gly | Levilimab | 10 | mg/ml |
| | Sodium acetate trihydrate | 1.742 | mg/ml |
| | Acetic acid glacial | to pH 5.0 | |
| | Mannitol | 45 | mg/ml |
| | L-glycine | 20 | mM |
| Ac + MANN + 100Gly | Levilimab | 10 | mg/ml |
| | Sodium acetate trihydrate | 1.742 | mg/ml |
| | Acetic acid glacial | to pH 5.0 | |
| | Mannitol | 45 | mg/ml |
| | L-glycine | 100 | mM |
| Ac + MANN + Arg + | Levilimab | 10 | mg/ml |
| Glu | Sodium acetate trihydrate | 1.742 | mg/ml |
| | Acetic acid glacial | to pH 5.0 | |
| | Mannitol | 45 | mg/ml |
| | L-arginine hydrochloride | 50 | mM |
| | monohydrate | | |
| | Sodium glutamate monohydrate | 50 | mM |
| Ac + MANN + Arg + | Levilimab | 10 | mg/ml |
| Glu + PLX188 | Sodium acetate trihydrate | 1.742 | mg/ml |
| | Acetic acid glacial | to pH 5.0 | |
| | Mannitol | 45 | mg/ml |
| | L-arginine hydrochloride | 50 | mM |
| | monohydrate | | |
| | Sodium glutamate monohydrate | 50 | mM |
| | Poloxamer 188 | 1 | mg/ml |
| Ac + MANN + Arg + | Levilimab | 10 | mg/ml |
| Glu + SBECD | Sodium acetate trihydrate | 1.742 | mg/ml |
| | Acetic acid glacial | to pH 5.0 | |
| | Mannitol | 45 | mg/ml |
| | L-arginine hydrochloride | 50 | mM |
| | monohydrate | | |
| | Sodium glutamate monohydrate | 50 | mM |
| | Cyclodextrin sulfobutyl ether | 30 | mg/ml |
| Ac + MANN + Arg + | Levilimab | 10 | mg/ml |
| Glu + HPBCD | Sodium acetate trihydrate | 1.742 | mg/ml |
| | Acetic acid glacial | to pH 5.0 | |
| | Mannitol | 45 | mg/ml |
| | L-arginine hydrochloride | 50 | mM |
| | monohydrate | | |
| | Sodium glutamate monohydrate | 50 | mM |
| | Hydroxypropyl-β-cyclodextrin | 30 | mg/ml |
| Ac + MANN + Met | Levilimab | 10 | mg/ml |
| | Sodium acetate trihydrate | 1.742 | mg/ml |
| | Acetic acid glacial | to pH 5.0 | |
| | Mannitol | 45 | mg/ml |
| | L-methionine | 100 | mM |
| Ac + PRO | Levilimab | 10 | mg/ml |
| | Sodium acetate trihydrate | 1.742 | mg/ml |
| | Acetic acid glacial | to pH 5.0 | |
| | L-Proline | 225 | mM |
| Ac + Arg + Glu | Levilimab | 10 | mg/ml |
| | Sodium acetate trihydrate | 1.742 | mg/ml |
| | Acetic acid glacial | to pH 5.0 | |
| | L-arginine hydrochloride | 100 | mM |
| | monohydrate | | |
| | Sodium glutamate monohydrate | 100 | mM |
| Ac + Arg + Glu + | Levilimab | 10 | mg/ml |
| PLX188 | Sodium acetate trihydrate | 1.742 | mg/ml |
| | Acetic acid glacial | to pH 5.0 | |
| | L-arginine hydrochloride | 100 | mM |
| | monohydrate | | |
| | Sodium glutamate monohydrate | 100 | mM |
| | Poloxamer 188 | 1 | mg/ml |
| Ac + Arg + Glu + | Levilimab | 10 | mg/ml |
| SBECD | Sodium acetate trihydrate | 1.742 | mg/ml |
| | Acetic acid glacial | to pH 5.0 | |
| | L-arginine hydrochloride | 100 | mM |
| | monohydrate | | |
| | Sodium glutamate monohydrate | 100 | mM |
| | Cyclodextrin sulfobutyl ether | 30 | mg/ml |
| Ac + Arg + Glu + | Levilimab | 10 | mg/ml |
| HPBCD | Sodium acetate trihydrate | 1.742 | mg/ml |
| | Acetic acid glacial | to pH 5.0 | |
| | L-arginine hydrochloride | 100 | mM |
| | monohydrate | | |
| | Sodium glutamate monohydrate | 100 | mM |
| | Hydroxypropyl-β-cyclodextrin | 30 | mg/ml |

Determination of Thermal Stability.

Thermal stability was studied according to technique 5 for 96 hours. The analysis was performed according to techniques 9-10. The results are shown in table 7. The results were analyzed using the heat-mapping tool in the Microsoft Excel software. The best results have a lighter shade of color.

### Determination of Colloidal Stability During Shaking.

Colloidal stability was studied according to method 6 for 96 hours. The results of the study are shown in Table 8. The results were analyzed using the heat-mapping tool in the Microsoft Excel software. The best results have a lighter shade of color.

Determination of Colloidal Stability During Freezing and Thawing.

Colloidal stability was studied according to method 7 for 96 hours. The results of the study are shown in Table 9. The results were analyzed using the heat-mapping tool in the Microsoft Excel software. The best results have a lighter shade of color.

The following formulation among the test samples may be distinguished by stability:

| Levilimab | 10 | mg/ml |
|---|---|---|
| Sodium acetate trihydrate | 1.742 | mg/ml |
| Acetic acid glacial | to pH 5.0 | |
| Sorbitol | 45 | mg/ml |
| Glycine | 100 | mM |

This pharmaceutical composition demonstrated sufficient stabilizing properties among all the test samples: a low level of absolute change in the acid-base profile during thermal stress, as well as a low decrease in the monomer content during thermal stress and freezing.

The formulation containing mannitol as an osmotic agent and glycine as a stabilizer demonstrated minimal monomer reduction during thermal stress.

The study did not reveal any significant advantages of using solubilizers for the thermal or colloidal stability of the protein as compared to using amino acids as a stabilizer.

### Example 4. Determination of Stability During Accelerated Storage

Studies of stability were conducted for the following formulations: two formulations comprising glycine as a stabilizer, sorbitol and mannitol as osmotic agents, and a formulation based on an arginine-acetate buffer system. Formulations containing arginine showed a fairly low level of changes in the acid-base profile during thermal stress and shaking, in addition, according to the literature, the use of arginine (Hong, T., et al. Current Protein and Peptide Science, 2018.19, 748-758) significantly reduces the viscosity of the pharmaceutical composition. The test formulations are shown in Table 10.

TABLE 10
Test formulations

| Ac + MANN + Gly | Levilimab | 20 | mg/ml |
|---|---|---|---|
| 20 mg/ml | Sodium acetate trihydrate | 0.436 | mg/ml |
| | Acetic acid glacial | to pH 5.0 | |
| | Mannitol | 23 | mg/ml |
| | Glycine | 7.5 | mg/ml |
| Ac + SORB + Gly | Levilimab | 20 | mg/ml |
| 20 mg/ml | Sodium acetate trihydrate | 0.436 | mg/ml |
| | Acetic acid glacial | to pH 5.0 | |
| | Sorbitol | 23 | mg/ml |
| | Glycine | 7.5 | mg/ml |
| Ac + Arg | Levilimab | 20 | mg/ml |
| 20 mg/ml | Sodium acetate trihydrate | 1.744 | mg/ml |
| | Acetic acid glacial | to pH 5.0 | |
| | Arginine hydrochloride | 100 | mM |
| Ac + MANN + Gly | Levilimab | 180 | mg/ml |
| 180 mg/ml | Sodium acetate trihydrate | 0.436 | mg/ml |
| | Acetic acid glacial | to pH 5.0 | |
| | Mannitol | 23 | mg/ml |
| | Glycine | 7.5 | mg/ml |
| Ac + SORB + Gly | Levilimab | 180 | mg/ml |
| 180 mg/ml | Sodium acetate trihydrate | 0.436 | mg/ml |
| | Acetic acid glacial | to pH 5.0 | |
| | Sorbitol | 23 | mg/ml |
| | Glycine | 7.5 | mg/ml |
| Ac + Arg 180 | Levilimab | 180 | mg/ml |
| mg/ml | Sodium acetate trihydrate | 1.744 | mg/ml |
| | Acetic acid glacial | to pH 5.0 | |
| | Arginine hydrochloride | 100 | mM |
| Ac + MANN + Gly | Levilimab | 220 | mg/ml |
| 220 mg/ml | Sodium acetate trihydrate | 0.436 | mg/ml |
| | Acetic acid glacial | to pH 5.0 | |
| | Mannitol | 23 | mg/ml |
| | Glycine | 7.5 | mg/ml |
| Ac + SORB + Gly | Levilimab | 220 | mg/ml |
| 220 mg/ml | Sodium acetate trihydrate | 0.436 | mg/ml |
| | Acetic acid glacial | to pH 5.0 | |
| | Sorbitol | 23 | mg/ml |
| | Glycine | 7.5 | mg/ml |
| Ac + Arg 220 | Levilimab | 220 | mg/ml |
| mg/ml | Sodium acetate trihydrate | 1.744 | mg/ml |
| | Acetic acid glacial | to pH 5.0 | |
| | Arginine hydrochloride | 100 | mM |

Accelerated Storage.

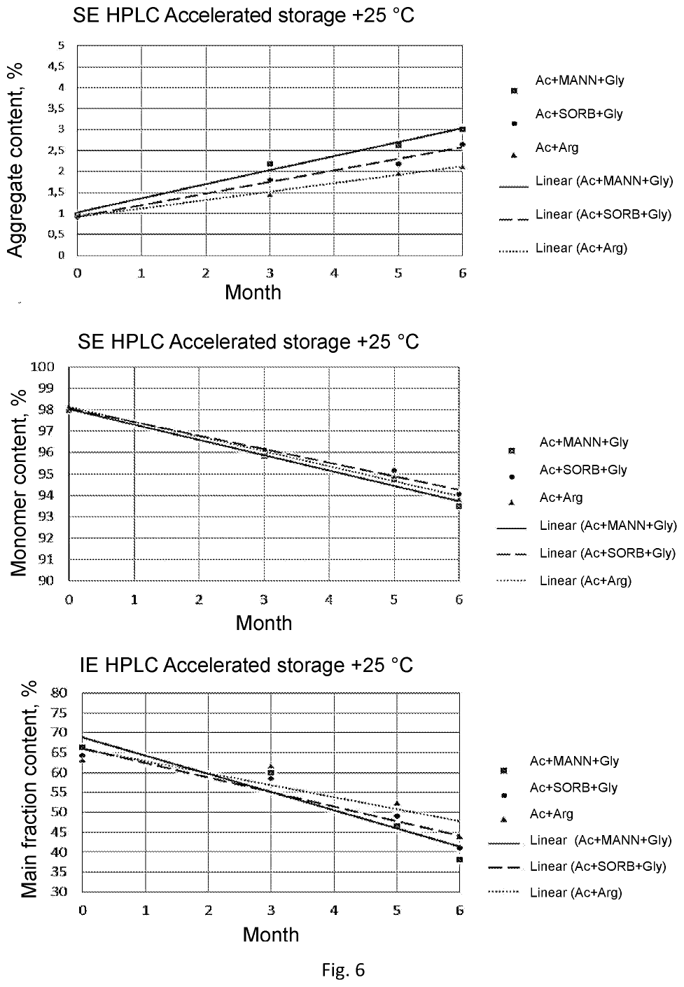

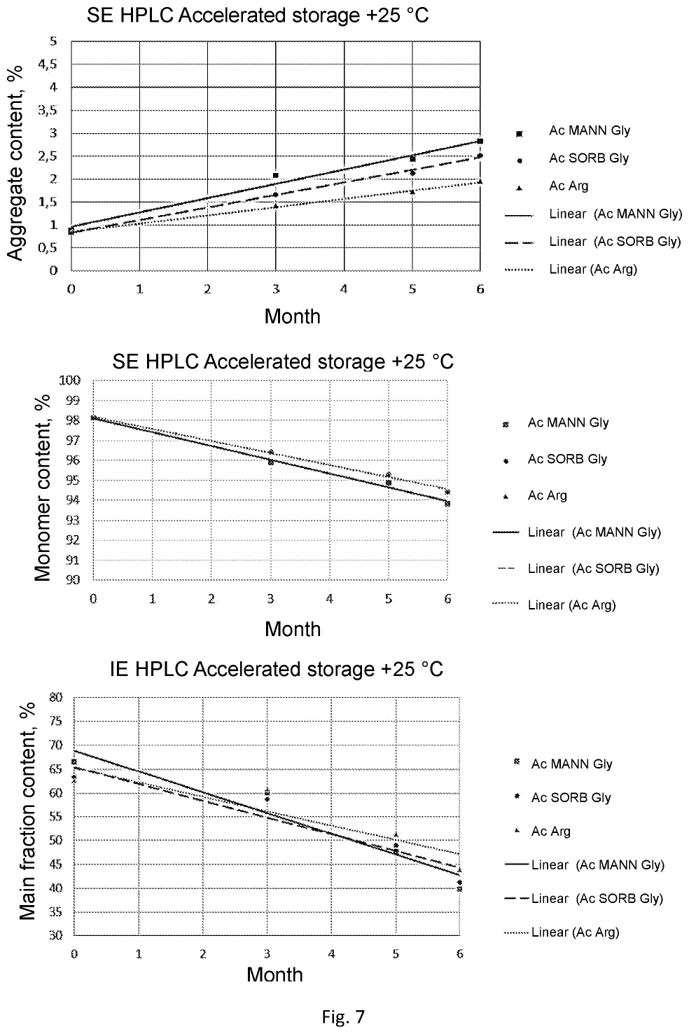

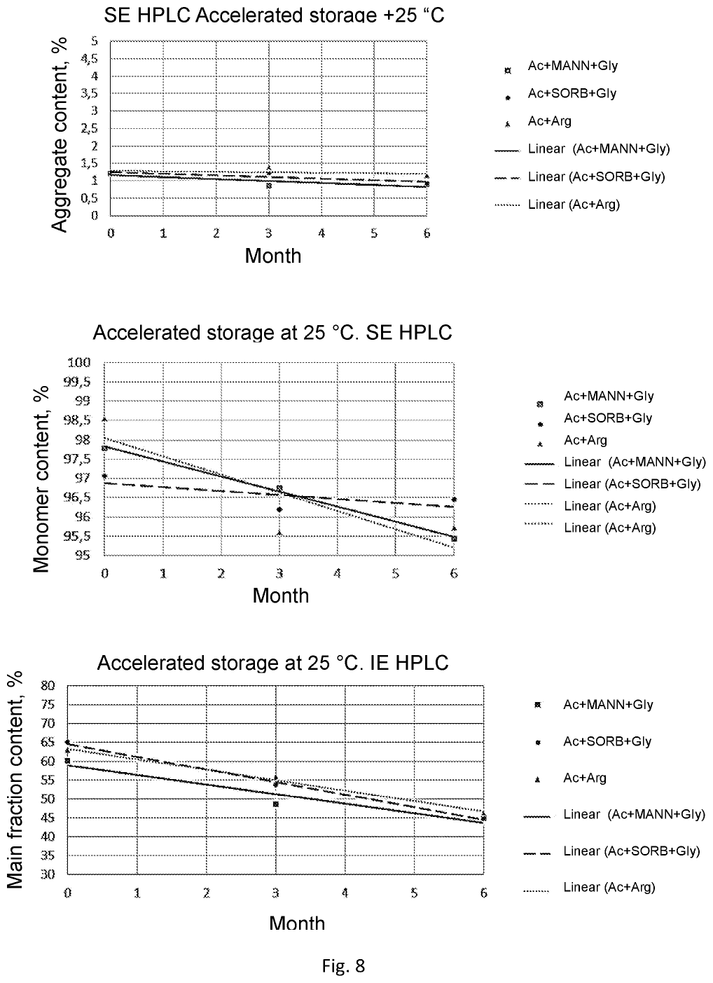

Pharmaceutical compositions comprising protein at a concentration of 20, 180 and 220 mg/ml were prepared by diafiltration according to technique 1 and placed for accelerated storage at a temperature of 25±2° C. in accordance with technique 8. The results of the study are shown in Table 11 and in FIGS. 6, 7 and 8.

TABLE 11
Results of stability study

| Formulation | | Initial | 3 | 5 | 6 | Abs. |
|---|---|---|---|---|---|---|
| name | Indicator | control | months | months | months | change |
| Ac + MANN + | pH | 5.91 | 5.90 | NA | 5.84 | NA |
| Gly 220 mg/ml | Osmolality, mOsm/kg | 362 | 360 | NA | 351 | NA |
| | Aggregate content, % | 0.97 | 2.18 | 2.63 | 3.01 | 2.04 |
| | Monomer content (SE | 97.98 | 95.82 | 94.73 | 93.51 | −4.47 |
| | HPLC), % | | | | | |
| | Acidic fractions, % | 22.50 | 32.81 | 44.18 | 47.76 | 25.26 |
| | Basic fractions, % | 66.27 | 59.97 | 46.54 | 38.23 | −28.04 |
| | Alkaline fractions, % | 11.23 | 7.22 | 9.28 | 14.01 | 2.78 |
| | Total change in acid- | NA | 20.26 | 43.36 | 56.08 | 56.08 |
| | base profile | | | | | |
| Ac + SORB + | pH | 5.52 | 5.49 | NA | 5.45 | NA |
| Gly 220 mg/ml | Osmolality, mOsm/kg | 375 | 361 | NA | 363 | NA |
| | Aggregate content, % | 0.91 | 1.80 | 2.19 | 2.64 | 1.73 |
| | Monomer content (SE | 98.03 | 96.13 | 95.15 | 94.07 | −4.04 |
| | HPLC), % | | | | | |
| | Acidic fractions, % | 22.83 | 30.47 | 39.70 | 43.55 | 20.72 |
| | Basic fractions, % | 64.12 | 58.62 | 49.03 | 41.12 | −23.00 |
| | Alkaline fractions, % | 13.05 | 10.91 | 11.27 | 15.33 | 2.28 |
| | Total change in acid- | NA | 15.28 | 33.74 | 46.00 | 46.00 |
| | base profile | | | | | |
| Ac + Arg | pH | 5.21 | 5.24 | NA | 5.32 | NA |
| 220 mg/ml | Osmolality, mOsm/kg | 320 | 312 | NA | 301 | NA |
| | Aggregate content, % | 0.94 | 1.45 | 1.96 | 2.11 | 1.15 |
| | Monomer content (SE | 98.06 | 96.41 | 95.22 | 94.03 | −4.03 |
| | HPLC), % | | | | | |
| | Acidic fractions, % | 22.15 | 24.36 | 30.33 | 37.36 | 15.21 |
| | Basic fractions, % | 63.22 | 61.77 | 52.43 | 43.92 | −19.30 |
| | Alkaline fractions, % | 14.63 | 13.87 | 17.24 | 18.72 | 4.09 |
| | Total change in acid- | NA | 4.42 | 21.58 | 38.60 | 38.60 |
| | base profile | | | | | |
| Ac + MANN + | pH | 5.85 | 5.94 | NA | 5.79 | NA |
| Gly 180 mg/ml | Viscosity, Pa · s | 0.011 | NA | NA | NA | NA |
| | Osmolality, mOsm/kg | 346 | 350 | NA | 332 | NA |
| | Aggregate content, % | 0.90 | 2.08 | 2.45 | 2.83 | 1.93 |
| | Monomer content (SE | 98.13 | 95.92 | 94.88 | 93.82 | −4.31 |
| | HPLC), % | | | | | |
| | Acidic fractions, % | 22.99 | 33.53 | 44.12 | 46.58 | 23.59 |
| | Basic fractions, % | 66.46 | 60.02 | 47.74 | 39.87 | −26.61 |
| | Alkaline fractions, % | 10.55 | 6.45 | 8.14 | 13.55 | 3.00 |
| | Total change in acid- | NA | 23.08 | 42.26 | 53.19 | 53.19 |
| | base profile | | | | | |
| | Relative specific | 94 | 100 | 88 | 91 | NA |
| | activity | | | | | |
| Ac + SORB + | pH | 5.43 | 5.43 | NA | 5.36 | NA |
| Gly 180 mg/ml | Viscosity, Pa · s | 0.012 | NA | NA | NA | NA |
| | Osmolality, mOsm/kg | 375 | 358 | NA | 367 | NA |
| | Aggregate content, % | 0.85 | 1.66 | 2.14 | 2.53 | 1.68 |
| | Monomer content (SE | 98.13 | 96.42 | 95.31 | 94.43 | −3.70 |
| | HPLC), % | | | | | |
| | Acidic fractions, % | 23.82 | 32.84 | 41.63 | 44.38 | 20.55 |
| | Basic fractions, % | 63.22 | 58.69 | 48.99 | 41.29 | −21.93 |
| | Alkaline fractions, % | 12.96 | 8.47 | 9.38 | 14.33 | 1.37 |
| | Total change in acid- | NA | 18.04 | 35.62 | 43.85 | 43.85 |
| | base profile | | | | | |
| | Relative specific | 92 | 103 | 88 | 100 | NA |
| | activity | | | | | |
| Ac + Arg | pH | 5.05 | 5.11 | NA | 5.03 | NA |
| 180 mg/ml | Viscosity, Pa · s | 0.008 | NA | NA | NA | NA |
| | Osmolality, mOsm/kg | 314 | 299 | NA | 291 | NA |
| | Aggregate content, % | 0.85 | 1.42 | 1.74 | 1.95 | 1.10 |
| | Monomer content (SE | 98.17 | 96.47 | 95.30 | 94.45 | −3.93 |
| | HPLC), % | | | | | |
| | Acidic fractions, % | 23.03 | 27.85 | 36.35 | 36.94 | 13.91 |
| | Basic fractions, % | 62.68 | 60.68 | 51.28 | 43.85 | −18.83 |
| | Alkaline fractions, % | 14.29 | 11.47 | 12.37 | 19.21 | 4.92 |
| | Total change in acid- | NA | 9.64 | 26.64 | 37.66 | 37.66 |
| | base profile | | | | | |
| | Relative specific | 97 | 94 | 85 | 115 | NA |
| | activity | | | | | |
| Ac + MANN + | pH | 5.27 | 5.30 | NA | 5.44 | NA |
| G]y 20 mg/ml | Osmolality, mOsm/kg | 260 | 258 | NA | 258 | NA |
| | Aggregate content, % | 1.22 | 0.86 | NA | 0.89 | −0.33 |
| | Monomer content (SE | 97.76 | 96.74 | NA | 95.44 | −2.34 |
| | HPLC), % | | | | | |
| | Acidic fractions, % | 28.33 | 33.99 | NA | 44.39 | 16.06 |
| | Basic fractions, % | 60.25 | 48.59 | NA | 45.17 | −15.08 |
| | Alkaline fractions, % | 11.42 | 17.42 | NA | 10.44 | −0.98 |
| | Total change in acid- | NA | 23.32 | NA | 32.12 | 32.12 |
| | base profile | | | | | |
| Ac + SORB + | pH | 5.09 | NA | NA | 5.46 | NA |
| Gly 20 mg/ml | Osmolality, mOsm/kg | 278 | NA | NA | 310 | NA |
| | Aggregate content, % | 1.19 | 1.22 | NA | 0.92 | −0.27 |
| | Monomer content (SE | 97.07 | 96.19 | NA | 96.45 | −0.62 |
| | HPLC), % | | | | | |
| | Acidic fractions, % | 24.91 | 34.89 | NA | 40.01 | 15.09 |
| | Basic fractions, % | 65.00 | 53.86 | NA | 44.97 | −20.03 |
| | Alkaline fractions, % | 10.09 | 11.25 | NA | 15.02 | 4.93 |
| | Total change in acid- | NA | 22.28 | NA | 40.07 | 40.07 |
| | base profile | | | | | |
| Ac + Arg | pH | 4.93 | NA | NA | 4.99 | NA |
| 20 mg/ml | Osmolality, mOsm/kg | 221 | NA | NA | 217 | NA |
| | Aggregate content, | 1.21 | 1.39 | NA | 1.14 | −0.07 |
| | HPLC), % | | | | | |
| | Acidic fractions, % | 24.69 | 31.23 | NA | 34.64 | 9.95 |
| | Basic fractions, % | 63.03 | 55.86 | NA | 46.37 | −16.66 |
| | Alkaline fractions, % | 12.28 | 12.91 | NA | 18.99 | 6.71 |
| | Total change in acid- | NA | 14.34 | NA | 33.32 | 33.32 |
| | base profile | | | | | |

All pharmaceutical compositions demonstrated an acceptable level of changes during accelerated storage.

The pharmaceutical composition comprising the acetate-arginine buffer system demonstrated an acceptable level of aggregation, as well as a low change in the acid-base profile during accelerated storage, both at a concentration of monoclonal antibody against the IL-6 receptor of 20 mg/ml, and at an increased concentration of up to 180-220 mg/ml.

The pharmaceutical composition comprising arginine demonstrated a reduced viscosity value.

The pharmaceutical composition comprising sorbitol as an osmotic agent demonstrated a low level of reduction in the monomer content during accelerated storage, both at a concentration of monoclonal antibody against the IL-6 receptor of 20 mg/ml, and at an increased concentration of 180-220 mg/ml.

Examples of studies using the aqueous pharmaceutical compositions of levilimab for treating IL-6-associated diseases have been provided. The aqueous pharmaceutical compositions used for these studies are described in Table 11.1:

TABLE 11.1

| | | | In a pre-filled | |
|---|---|---|---|---|
| | In 1.0 _{MJI} | | syringe (0.9 ml) | |
| Levilimab | 180 | mg | 162 | mg |
| Sodium acetate trihydrate | 0.436 | mg | 0.392 | mg |
| Glycine | 7.5 | mg | 6.8 mg or 6.75 mg | |
| Mannitol | 23.0 | mg | 20.7 | mg |

| | | |
|---|---|---|
| Glacial acetic acid | To pH 5.0 | To pH 5.0 |
| Water for injections | To 1.0 ml | To 0.9 ml |

It has been shown that different doses of the pharmaceutical compositions of levilimab according to the present invention and/or the drug Levilimab (also referred to in examples 5 and 6 as LVL and BCD-089) including the aqueous pharmaceutical compositions of levilimab according to the present invention, described in Table 11.1 are suitable for treating the corresponding IL-6-associated diseases.

### Example 5. International, Multi-Center, Comparative, Randomized, Double-Blind Placebo-Controlled Clinical Trial of Efficacy and Safety of Levilimab in Different Dosage Regimens in Subjects with Active Rheumatoid Arthritis

The study included a screening period, main period (during which the subjects received therapy in the blinded fashion), the open-label period (during which the subjects received therapy in the unblinded fashion), and the follow-up period:
Screening period (28-42 days)
Main trial period: Week 0-Week 12
Open-label period (Week 12-Week 52)
Follow-up period (4 calendar weeks up to week 56).

The study included men and women aged 18-80 years inclusive, with definitely diagnosed rheumatoid arthritis meeting the 2010 ACR criteria and diagnosed at least 6 months prior to the date of signing the informed consent, who received methotrexate therapy for at least three months and for at least the last 4 weeks at a stable dose, while maintaining the disease activity at the time of signing the informed consent and maintaining the rheumatoid arthritis activity despite methotrexate therapy conducted during the screening period (4-6 weeks) without significant concomitant pathology, in accordance with the criteria for inclusion and non-inclusion in the trial.

The final population in this trial was 105 subjects:
35 subjects were randomized into an arm receiving levilimab at a dose of 162 mg subcutaneously once a week (LVL QW group);
35 subjects were randomized into an arm receiving levilimab at a dose of 162 mg subcutaneously once in 2 weeks (LVL Q2W group);
35 subjects were randomized into an arm (Placebo/LVL Q2W arm) receiving placebo during the first 12 weeks of treatment. Starting from week 12, subjects in this arm received levilimab therapy, at a dose of 162 mg subcutaneously once every 2 weeks until week 52 of the trial.

Test Medicinal Product:

INN: levilimab, monoclonal antibody against interleukin-6 receptor, injectable solution, 180 mg/ml.

Dosage: 162 mg/0.9 ml

Route of administration: subcutaneous.

Duration of treatment with levilimab:
LVL QW arm: 53 weeks (Week 0-Week 52). Subjects in this arm could receive a maximum of 53 injections of levilimab.
LVL Q2W ar: 53 weeks (Week 0-Week 52). Subjects in this arm could receive a maximum of 27 injections of the drug levilimab.
Placebo/LVL Q2W arm: 41 weeks (Week 12-Week 52). Subjects in this arm could receive a maximum of 21 injections of the drug levilimab over the period in question.

Endpoints for assessing the efficacy of the main trial period:

Primary Endpoint:
The proportion of subjects with rheumatoid arthritis in each arm who, by week 12 following the first administration of BCD-089/placebo, achieved an improvement in the course of the disease, corresponding to ACR20.

Additional endpoints for the main trial period:
The proportion of subjects with rheumatoid arthritis in each arm who achieved an improvement in the course of the disease, corresponding to ACR20, at week 4 and 8 from the first administration of BCD-089/placebo.
The proportion of subjects with rheumatoid arthritis in each arm who achieved an improvement in the course of the disease, corresponding to ACR50/70, at week 4, 8 and 12 (DAS28-CRP(4) following the first administration of BCD-089/placebo.
The proportion of subjects in each arm with low RA activity according to the DAS28-CRP(4) index (DAS28-CRP(4)<3.2) CDAI (CDAI≤10, SDAI (SDAI≤11) at week 4, 8 and 12 following the first administration of BCD-089/placebo.
Changes in the DAS28-CRP(4), CDAI, and SDAI indices at week 12 as compared to the baseline values.
Changes in the erythrocyte sedimentation rate at week 12 of therapy as compared to baseline values.

Additional endpoints for the open-label trial period
The proportion of subjects with rheumatoid arthritis who achieved an improvement in the course of the disease, corresponding to ACR20/50/70, at week 16, 24, 36, 48 and 52 following the first administration of BCD-089.
The proportion of subjects with low RA activity according to the DAS28-CRP(4) index (DAS28-CRP(4)<3.2) CDAI (CDAI≤10, SDAI (SDAI≤11) at week 16, 24, 36, 48 and 52 following the first administration of BCD-089.
Changes in the DAS28-CRP(4), CDAI, and SDAI indices as compared to the baseline values.
The proportion of subjects who achieved remission according to the ACR/EULAR 2011 criteria at week 24, 36, 48 and 52 of BCD-089 therapy.
Patient-reported assessment of quality of life before treatment, 24 and 52 weeks following the first administration of BCD-089 according to the SF36 questionnaire.
Changes in the erythrocyte sedimentation rate as compared to the baseline values.
Radiographic characteristics of the affected joints 52 weeks following the first administration of BCD-089.
Mean change of the total score according to Sharp-van der Heijde (1989) modified assessment method.
Proportion of subjects with an increased radiographic stage of rheumatoid arthritis (assessed by the Steinbrocker's method).

Endpoint for Pharmacodynamics Assessment

Secondary Endpoints

Pharmacodynamics was analyzed by determining the below analytes' concentration in serum by solid-phase ELISA:
soluble interleukin-6 receptor
C-reactive protein
IL-6
TNFα

Secondary Endpoints
Eₘᵢₙ (minimum concentration of CRP in serum).
ETₘᵢₙ (time to reach the minimum concentration of CRP).
AUEC₀₋ₜ (the area under the curve “concentration for CRP, sIL-6R, TNFα and IL-6-time” (AUC—area under curve) from the moment of product administration to the last concentration measurement).
Eₘₐₓ (maximum concentration of SIL-6R in serum).
ETₘₐₓ (time to reach the maximum concentration of sIL-6R).

Endpoints for Safety Assessment
Proportion of subjects with adverse events, including serious adverse events, in each arm.
Proportion of subjects with serious adverse events in each arm.
Proportion of subjects with grade 3-4 adverse events in each arm.
Proportion of subjects with grade 3-4 neutropenia in each arm.
Proportion of subjects in each arm with adverse events characteristic of IL-6 receptor inhibitors:
Increased ALT/AST activity;
Leukopenia/Neutropenia;
Thrombocytopenia;
Upper respiratory tract infections; phlegmon; pneumonia; infections with Herpes Simplex type 1 and Herpes Zoster; diverticulitis;
Increase in total cholesterol/HDL/LDL/triglycerides.
Proportion of subjects who discontinued the trial early due to AE/SAE in each arm.

Endpoint for Immunogenicity Assessment

Main Trial Period
Proportion of subjects with detected binding and/or neutralizing antibodies to BCD-089 product at week 12.

Open-Label Trial Period
Proportion of subjects with detected binding and/or neutralizing antibodies to BCD-089 product at weeks 24 and 52.

Results of Efficacy Assessment:

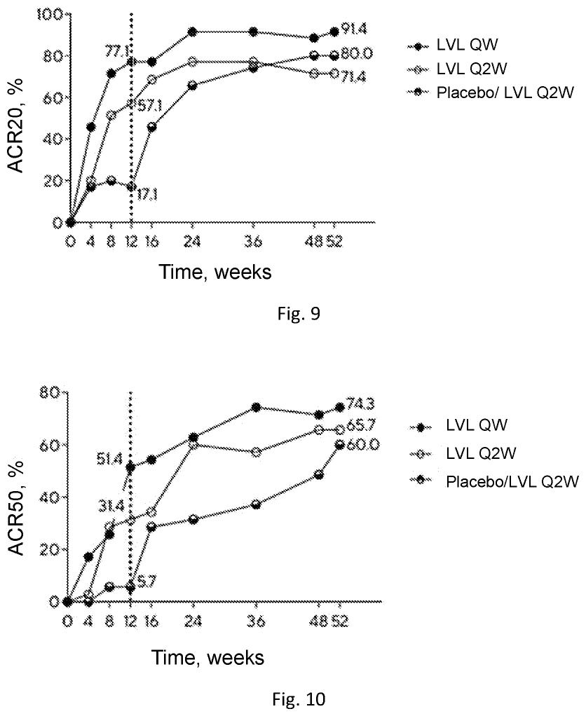

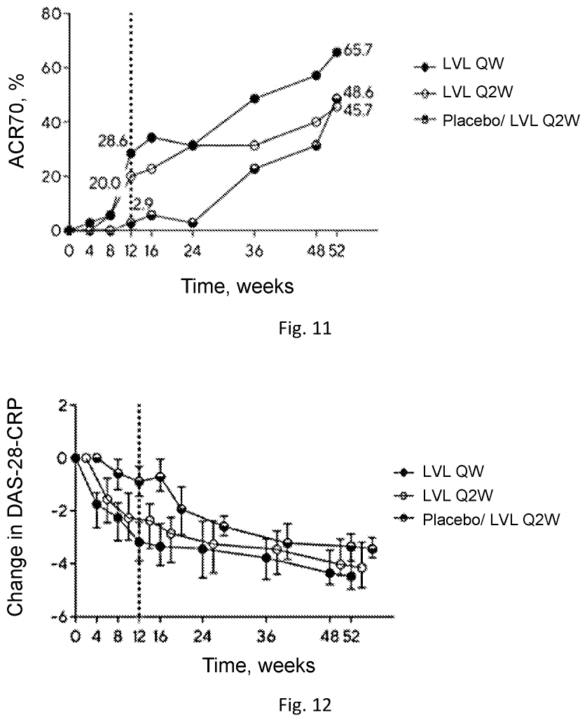

During the use of levilimab throughout the year we observed a continued increase in the number of subjects with improvements in the course of the disease. At the same time, the least pronounced response corresponding to ACR20 was achieved by the majority of subjects during the first 24 weeks of therapy, and, later on, the increase in the number of responders was due to the subjects' achievement of ACR50 and, to a greater extent, of ACR70 (FIGS. 9, 10, 11).

Starting from week 4 of therapy, the proportion of subjects with low RA activity in the LVL QW arm was numerically higher as compared to the LVL Q2W arm for CDAI and SDAI indices, whereas for DAS28-CRP(4), the differences reached statistical significance at week 12. The DAS-28-CRP(4), CDAI, and SDAI indices showed a distinct positive trend throughout 52 weeks of therapy, reflecting a decrease in the severity of clinical symptoms of RA. During the first 12 weeks of therapy, changes in the indices were numerically more pronounced in the LVL QW arm as compared to the LVL Q2W arm, whereas for the DAS28-CRP index(4), the differences reached statistical significance also by week 12 (FIG. 12). In general, the dynamics of RA activity, reflected both by the proportions of subjects with low activity and changes in the indices, indicates a higher rate of clinical response in the LVL QW arm.

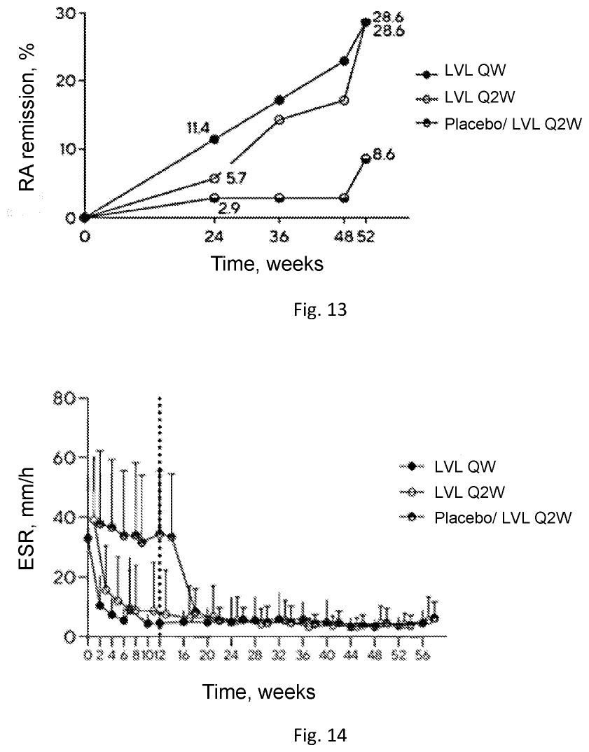

The frequency of achieving remission in the course of RA (according to ACR/FULAR 2011) was comparable at week 52 of therapy in the LVL QW and LVL Q2W arm, but despite the lack of statistical significance of differences, the LVL QW arm had numerically higher values of the indicator at weeks 24, 36 and 48 as compared to that of the LVL Q2W arm, which also confirms a higher rate of clinical response in the arm of subjects who used the drug once a week (FIG. 13).

Analysis of changes in ESR relative to the baseline on the background of levilimab therapy revealed that the LVL QW and LVL Q2W arms, following the first administration of the test drug, showed a significant decrease in the erythrocyte sedimentation rate, which reached minimum values during the first 2-4 weeks of therapy and did not change significantly thereafter, remaining minimal until the end of the trial (FIG. 14).

The results of assessment of the physical (PH) and mental health (MH) components of quality of life by the SF-36 questionnaire showed that levilimab therapy is accompanied by an improvement in the patient-reported assessment of both the physical and mental health components of quality of life.

Assessment of radiographic changes in the joints (by the Sharp-van der Heijde modified method) showed that absolute values of the indicator did not differ statistically significantly between screening and week 52 on the background of levilimab therapy. However, the analysis of changes in the indicator revealed a statistically significant difference between the LVL QW and LVL Q2W arms (p=0.0494) at week 52. The LVL QW arm demonstrated no changes in the total score during the year, whereas the LVL Q2W arm showed increased radiographic changes in 3 subjects. Increase in the radiographic stage of RA by the Steinbrocker's method was observed only in one subject of the Placebo/LVL Q2W arm.

Assessment of the proportion of subjects with an increase in the radiographic stage of rheumatoid arthritis did not reveal subjects with a progression of the radiographic stage by the Steinbrocker's method in the LVL QW and LVL Q2W arms.

The data on primary endpoint efficacy enables accepting the hypothesis of superior efficacy of levilimab versus placebo in all test populations (PP and ITT), both when using the once-a-week and once-in-2-week regimens, and, accordingly, concluding that the both test dosage regimens of levilimab were effective in subjects with active rheumatoid arthritis and that the trial achieved its objective. Further, more frequent administration of the test drug (once a week for a year) showed slightly better efficacy as compared to the once-in-2-week regimen, both in terms of the time of achievement of the objective and the magnitude of the response to therapy.

Data analysis on the safety of levilimab in subjects with active rheumatoid arthritis throughout 1 year showed that the levilimab product at a dose of 162 mg has a favorable safety profile and low immunogenicity, regardless of the mode of administration.

In the course of treatment with the test drug we observed a significant change in the serum concentrations of pharmacodynamic markers, i.e., increased concentration of sIL-6R, IL-6 and decreased concentration of CRP. The once-a-week dosage regimen provides a significantly more pronounced increase in the concentration of sIL-6R (characteristic of the group of SIL-6R inhibitors) and is characterized by a tendency to a faster and more pronounced decrease in the CRP concentration. In general, the dynamics of pharmacodynamic markers indicate a highly effective neutralization of the soluble IL-6 receptor by the levilimab product, which, in turn, is manifested in a rapid and pronounced decrease in the serum CRP concentration, reflecting the effective suppression of the inflammatory process in subjects with active rheumatoid arthritis. Further, the administration of levilimab in the once-a-week regimen demonstrated greater efficacy with respect to pharmacodynamic markers as compared to the once-in-two-week regimen.

### Example 6. Assessment of Pharmacodynamics

Serum concentrations of the soluble interleukin-6 receptor (sIL-6R) and C-reactive protein (CRP) were used as pharmacodynamic markers in this study.

The analysis of pharmacodynamic parameters included data from 104 subjects: 35 subjects who received once-a-week s/c administration of levilimab (BCD-089 QW arm), 34 subjects who received once-in-two-weeks s/c administration of levilimab (BCD-089 Q2W arm), and 35 subjects of the Placebo arm.

1 subject from the BCD-089 Q2W arm who withdrew informed consent to participate in the trial at visit 1 prior to the first administration of the test drug was excluded from the pharmacodynamic analysis.

Assessment of Concentrations of Soluble IL-6 Receptor (sIL-6R)

The concentration of sIL-6R in serum (it reflects the blocking of the receptor by the test drug, characteristic of drugs of the sIL6R inhibitor group) increased in serum of subjects of the both test drug arms and reached the highest values (Eₘₐₓ) in the BCD-089 QW arm (3240960 [1937060-4108080] pg/ml after 2016 [1344-2016] h. The BCD-089 Q2W arm showed Eₘₐₓ of 1835030 [1536920-3020400] pg/ml after 2016 [1344; 2016] hours. The Placebo arm showed no increase in sIL-6R concentration, Eₘₐₓ was 228440 [168822-367380] pg/ml after 96 [48-504] hours. Statistically significant differences were revealed both between the test drug arms and placebo arm (p<0.0001; Kruskal-Wallis test), and between the test drug arms (p=0.0112; Kruskal-Wallis test).

The detailed results of the statistical analysis of the SIL-6R concentration are shown in the table below.

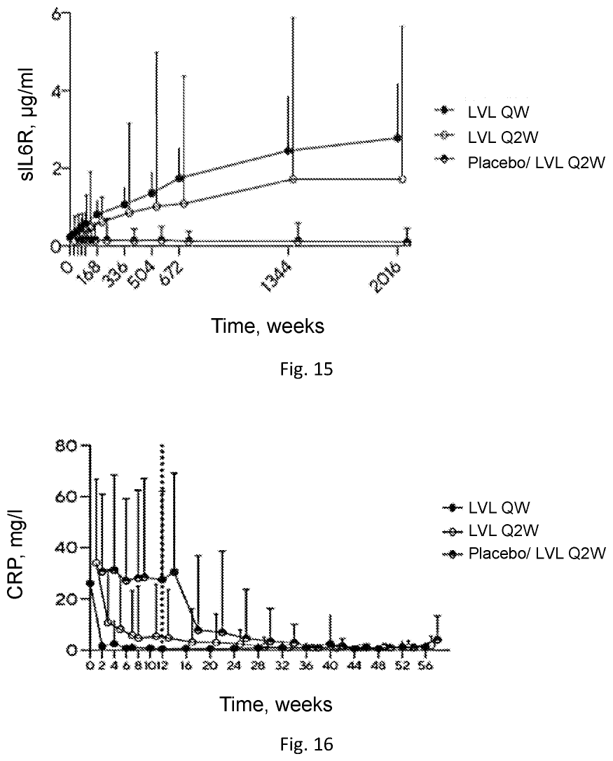

The administered BCD-089 dose determined the values of area under the concentration/time curve (AUEC_{0-last) which reached significantly high values in the BCD-}089 QW arm and with BCD-089 Q2W and Placebo (p<0.0001; Kruskal-Wallis test). Further, the differences between the test arms also showed significant differences (p=0.0066; Kruskal-Wallis test) (FIG. 15).

TABLE 12
Pharmacodynamic indicators of serum
SIL-6 concentrations in the test arms

| Indicator | Parameter | BCD-089 QW | BCD-089 Q2W | Placebo | P-value |
|---|---|---|---|---|---|
| AUEC₍₀₋ₗₐₛₜ₎ | Amount | 35 | 34 | 35 | <0.0001* |
| (pg/ml) · h | Mean | 3877477378.6 | 3057063565.8 | 301313346.86 | |
| | value | | | | |
| | Geometric | 3642811867.1 | 2587908634.4 | 245387910.79 | |
| | mean | | | | |
| | Median | 3635343120 | 2525842488 | 251449656 | |
| | Minimum | 1564731384 | 319388184 | 81350940 | |
| | Maximum | 8077128000 | 7928934000 | 832511280 | |
| | L. | 2929609584 | 1785569760 | 138218772 | |
| | quartile | | | | |
| | Up. | 4739909412 | 3865588620 | 440677152 | |
| | quartile | | | | |
| | St. Dev. | 1446141280.7 | 1821875372.7 | 195707647.11 | |
| | CV. % | 37.296 | 59.596 | 64.952 | |
| Eₘₐₓ | Amount | 35 | 34 | 35 | <0.0001* |
| pg/ml | Mean | 3279889.143 | 2396267.647 | 273365.143 | |
| | value | | | | |
| | Geometric | 2936587.689 | 2070172.48 | 239274.305 | |
| | mean | | | | |
| | Median | 3240960 | 1835030 | 228440 | |
| | Minimum | 1154240 | 881020 | 79488 | |
| | Maximum | 7154640 | 5869560 | 704160 | |
| | L. | 1937060 | 1536920 | 168822 | |
| | quartile | | | | |
| | Up. | 4108080 | 3020400 | 367380 | |
| | quartile | | | | |
| | St. Dev. | 1590726.089 | 1423477.899 | 147917.298 | |
| | CV. % | 48.499 | 59.404 | 54.11 | |
| ETₘₐₓ | Amount | 35 | 34 | 35 | <0.0001* |
| hour | Mean | 1598.4 | 1680 | 318.857 | |
| | value | | | | |
| | Geometric | 1492.272 | 1564.577 | 0 | |
| | mean | | | | |
| | Median | 2016 | 2016 | 96 | |
| | Minimum | 504 | 336 | 0 | |
| | Maximum | 2016 | 2016 | 2016 | |
| | L. | 1344 | 1344 | 48 | |
| | quartile | | | | |
| | Up. | 2016 | 2016 | 504 | |
| | quartile | | | | |
| | St. Dev. | 497.988 | 494.579 | 450.865 | |
| | CV. % | 31.155 | 29.439 | 141.4 | |

Note:
*Kruskal-Wallis test

Assessment of C-Reactive Protein Concentration

The C-reactive protein concentrations in serum of the subjects of the arms showed a distinct decrease in the course of the treatment. The maximum reduction was detected in the BCD-089 QW arm, Eₘᵢₙ, was 0 [0; 404] ng/ml and was achieved after 672 [336; 1344] hours. The corresponding values in the BCD-089 Q2W arm were 72 [0; 421] ng/ml, which were reached after 1344 [504; 2016] hours, whereas no significant differences were observed between the arms (p>0.05).

The minimum CRP concentration in the Placebo arm was 1421 [1087; 2266] ng/ml and was observed after 336 [96; 672] hours. The Eₘᵢₙ and ETₘᵢₙ indicators in the placebo arm were significantly different from the corresponding indicators in the both test drug arms (FIG. 16).

TABLE 13
Pharmacodynamic indicators of
C-reactive protein in subjects' serum

| | | | | | |
|---|---|---|---|---|---|
| Indicator | Parameter | BCD-089 QW | BCD-089 Q2W | Placebo | P-value |

| | | | | | |
|---|---|---|---|---|---|
| AUEC₍₀₋ₗₐₛₜ₎ | Amount | 35 | 34 | 35 | <0.0001⁴ |
| (pg/ml) · h | Mean value | 1154300.229 | 1629603.424 | 4462112.914 | |
| | Geometric | 887762.324 | 1240190.492 | 4129894.127 | |
| | mean | | | | |
| | Median | 1204572 | 1383354 | 4620132 | |
| | Minimum | 89676 | 130068 | 1506708 | |
| | Maximum | 2647728 | 4877364 | 7380132 | |
| | L. | 420144 | 786024 | 3022260 | |
| | quartile | | | | |
| | Up. | 1701996 | 2155956 | 5696520 | |
| | quartile | | | | |
| | St. Dev. | 689530.689 | 1132721.345 | 1664872.408 | |
| | CV. % | 59.736 | 69.509 | 37.311 | |
| Emin | Amount | 35 | 34 | 35 | <0.0001⁴ |
| ng/ml | Mean value | 177.543 | 256.353 | 1600.829 | |
| | Geometric | 0 | 0 | 0 | |
| | mean | | | | |
| | Median | 0 | 72 | 1421 | |
| | Minimum | 0 | 0 | 0 | |
| | Maximum | 805 | 1381 | 3363 | |
| | L. | 0 | 0 | 1087 | |
| | quartile | | | | |
| | Up. | 404 | 421 | 2266 | |
| | quartile | | | | |
| | St. Dev. | 257.904 | 340.909 | 772.04 | |
| | CV. % | 145.263 | 132.984 | 48.228 | |
| ETmin | Amount | 35 | 34 | 35 | <0.0001⁴ |
| hour | Mean value | 905.143 | 1210.588 | 544.457 | |
| | Geometric | 697.386 | 1000.641 | 268.995 | |
| | mean | | | | |
| | Median | 672 | 1344 | 336 | |
| | Minimum | 96 | 168 | 24 | |
| | Maximum | 2016 | 2016 | 2016 | |
| | L. | 336 | 504 | 96 | |
| | quartile | | | | |
| | Up. | 1344 | 2016 | 672 | |
| | quartile | | | | |
| | St. Dev. | 594.912 | 648.396 | 584.171 | |
| | CV. % | 65.726 | 53.56 | 107.294 | |

In the course of treatment with the test drug we observed a significant change in the serum concentrations of pharmacodynamic markers, i.e. increased sIL-6R concentration and decreased CRP concentration.

The resulting values of pharmacodynamic indicators displayed statistically significant differences with those obtained in the Placebo arm. Furthermore, the parameters characterizing the concentration of sIL-6R (increased concentration of which reflects the blocking of the receptor by the test drug and is characteristic of drugs of the sIL6R inhibitor group) also had significant differences between the test drug arms. Further, there was a tendency to a more rapid and pronounced decrease in the CRP concentration in the BCD-089 qw arm as compared to the BCD-089 Q2W arm, however, these differences were not significant.

In general, the dynamics of pharmacodynamic markers indicate a highly effective neutralization of the soluble IL-6 receptor by the BCD-089 product, which, in turn, is manifested in a rapid and pronounced decrease in the serum CRP concentration, indicating the effective suppression of the inflammatory response in subjects with active rheumatoid arthritis. The administration of the BCD-089 product in the once-a-week regimen demonstrated better efficacy with respect to pharmacodynamic markers as compared to the once-in-two-week regimen.

The use of anti-IL-6R therapy is known to be effective in cytokine release syndrome and adult (acute) respiratory distress syndrome. Considering the resulting data on the pharmacodynamics of levilimab, which shows its ability to efficiently block IL-6 signaling, it can be concluded that levilimab will be effective in the treatment of cytokine release syndrome (CRS) and adult (acute) respiratory distress syndrome (ARDS).

CRS has been identified as the main cause of mortality in subjects with SARS-CoV, MERS-CoV, and COVID-19, where increased interleukin 6 (IL-6) levels observed in these subjects are associated with the C-reactive protein (CRP) levels, respiratory failure, ARDS, and adverse clinical outcomes.

1. An aqueous pharmaceutical composition of levilimab comprising:
(i) 5-220 mg/ml levilimab;
(ii) 0.4-1.8 mg/ml sodium acetate trihydrate;
(iii) 20-50 mg/ml polyol;
(iv) 5-10 mg/ml glycine; and
(v) acetic acid to pH 4.5-6.5.

2. The aqueous pharmaceutical composition according to claim 1, wherein levilimab is present at a concentration of 5-40 mg/ml.

3. The aqueous pharmaceutical composition according to claim 1, wherein levilimab is present at a concentration of 20 mg/ml.

4. The aqueous pharmaceutical composition according to claim 1, wherein levilimab is present at a concentration of 180-220 mg/ml.

5. The aqueous pharmaceutical composition according to claim 1, wherein levilimab is present at a concentration of 180 mg/ml.

6. The aqueous pharmaceutical composition according to any of claims 1-5, wherein said sodium acetate trihydrate is present at a concentration of 0.4-1.0 mg/ml.

7. The aqueous pharmaceutical composition according to any of claims 1-5, wherein said sodium acetate trihydrate is present at a concentration of 0.4-0.5 mg/ml.

8. The aqueous pharmaceutical composition according to any of claims 1-5, wherein said sodium acetate trihydrate is present at a concentration of 0.436 mg/ml.

9. The aqueous pharmaceutical composition according to any of claims 1-8, wherein said polyol is present at a concentration of 20-26 mg/ml.

10. The aqueous pharmaceutical composition according to any of claims 1-8, wherein said polyol is present at a concentration of 23 mg/ml.

11. The aqueous pharmaceutical composition according to any of claims 1-8, wherein said polyol is selected from mannitol or sorbitol or a combination thereof.

12. The aqueous pharmaceutical composition according to any of claims 1-11, wherein said glycine is present at a concentration of 7-8 mg/ml.

13. The aqueous pharmaceutical composition according to any of claims 1-11, wherein said glycine is present at a concentration of 7.5 mg/ml.

14. The aqueous pharmaceutical composition according to any of claims 1-13, wherein said acetic acid is added to pH 5.0.

15. The aqueous pharmaceutical composition according to claim 1 comprising:
(i) 20 mg/ml levilimab;
(ii) 0.436 mg/ml sodium acetate trihydrate;
(iii) 23 mg/ml polyol selected from mannitol or sorbitol;
(iv) 7.5 mg/ml glycine; and
(v) acetic acid to pH 5.0.

16. The aqueous pharmaceutical composition according to claim 1 comprising:
(i) 180 mg/ml levilimab;
(ii) 0.436 mg/ml sodium acetate trihydrate;
(iii) 23 mg/ml polyol selected from mannitol or sorbitol;
(iv) 7.5 mg/ml glycine; and
(v) acetic acid to pH 5.0.

17. An aqueous pharmaceutical composition of levilimab comprising:
(i) 5-220 mg/ml levilimab;
(ii) 0.4-1.8 mg/ml sodium acetate trihydrate;
(iii) 10-32 mg/ml arginine hydrochloride; and
(iv) acetic acid to pH 4.5-6.5.

18. The aqueous pharmaceutical composition according to claim 17, wherein levilimab is present at a concentration of 5-40 mg/ml.

19. The aqueous pharmaceutical composition according to claim 17, wherein levilimab is present at a concentration of 20 mg/ml.

20. The aqueous pharmaceutical composition according to claim 17, wherein levilimab is present at a concentration of 180-220 mg/ml.

21. The aqueous pharmaceutical composition according to claim 17, wherein said levilimab is present at a concentration of 180 mg/ml.

22. The aqueous pharmaceutical composition according to any of claims 17-21, wherein said sodium acetate trihydrate is present at a concentration of 1.7-1.8 mg/ml.

23. The aqueous pharmaceutical composition according to any of claims 17-21, wherein said sodium acetate trihydrate is present at a concentration of 1.744 mg/ml.

24. The aqueous pharmaceutical composition according to any of claims 17-23, wherein said arginine hydrochloride is present at a concentration of 18-24 mg/ml.

25. The aqueous pharmaceutical composition according to any of claims 17-23, wherein said arginine hydrochloride is present at a concentration of 21.1 mg/ml.

26. The aqueous pharmaceutical composition according to any of claims 17-25, wherein said acetic acid is added to pH 5.0.

27. The aqueous pharmaceutical composition according to claim 17 comprising:
(i) 20 mg/ml levilimab;
(ii) 1.744 mg/ml sodium acetate trihydrate;
(iii) 21.1 mg/ml arginine hydrochloride; and
(iv) acetic acid to pH 5.0.

28. The aqueous pharmaceutical composition according to claim 17 comprising:
(i) 180 mg/ml levilimab;
(ii) 1.744 mg/ml sodium acetate trihydrate;
(iii) 21.1 mg/ml arginine hydrochloride; and
(iv) acetic acid to pH 5.0.

29. An aqueous pharmaceutical composition of levilimab comprising:
(i) 162 mg of levilimab;
(ii) 0.392 mg of sodium acetate trihydrate;
(iii) 20.7 mg of polyol selected from mannitol or sorbitol;
(iv) 6.75 mg of glycine;
(v) acetic acid to pH 5.0; and
(vi) water for injections to 0.9 ml.

30. An aqueous pharmaceutical composition of levilimab comprising:
(i) 162 mg of levilimab;
(ii) 1.57 mg of sodium acetate trihydrate;
(iii) 18.99 mg of arginine hydrochloride;
(iv) acetic acid to pH 5.0; and
(v) water for injections to 0.9 ml.

31. The aqueous pharmaceutical composition according to any of claims 1-30, wherein said acetic acid is glacial acetic acid.

32. The aqueous pharmaceutical composition according to any of claims 1-31, wherein said composition is intended for parenteral administration.

33. The aqueous pharmaceutical composition according to any of claims 1-31, wherein said composition is intended for intramuscular, intravenous, or subcutaneous administration.

34. The aqueous pharmaceutical composition according to any of claims 1-31, wherein said composition is present in a vial.

35. The aqueous pharmaceutical composition according to claim 34, wherein said vial is a glass vial or plastic vial.

36. The aqueous pharmaceutical composition according to any of claims 34-35, wherein said vial has a volume of 4-20 ml.

37. The aqueous pharmaceutical composition according to claim 31 wherein said vial has a volume of 4 ml, 10 ml or 20 ml.

38. The aqueous pharmaceutical composition according to any of claims 1-31, wherein said composition is present in a syringe or autoinjector.

39. The aqueous pharmaceutical composition according to claim 38, wherein said syringe or autoinjector is a glass syringe or autoinjector or plastic syringe or autoinjector.

40. The aqueous pharmaceutical composition according to any of claims 38-39, wherein said syringe or autoinjector has a capacity of 1 ml.

41. The aqueous pharmaceutical composition according to any of claims 1-31, wherein said composition is present in a pre-filled syringe or in a pre-filled autoinjector.

42. The aqueous pharmaceutical composition according to claim 41, wherein said pre-filled syringe or pre-filled autoinjector is a glass pre-filled syringe or autoinjector or plastic pre-filled syringe or autoinjector.

43. The aqueous pharmaceutical composition according to any of claims 41-42, wherein said pre-filled syringe or pre-filled autoinjector has a capacity of 1 ml.

44. The use of the aqueous pharmaceutical composition of levilimab according to any of claims 1, 17, 29, 30 for treating or preventing an IL6R-associated disease or disorder.

45. The use according to claim 44, wherein the IL6R-associated disease or disorder is selected from:
rheumatoid arthritis, juvenile chronic arthritis, scleroderma, graft versus host disease, organ transplant rejection, acute or chronic immune disease associated with organ transplantation, cachexia, adult (acute) respiratory distress syndrome, Still's disease, systemic scleroderma, Sjogren's syndrome, Takayasu's disease/arteritis, cytokine therapy associated disorders, cytokine release syndrome, iridocyclitis, uveitis, optic neuritis, optical neuromyelitis, juvenile rheumatoid arthritis, giant cell arteritis, polyarticular juvenile idiopathic arthritis, systemic-onset juvenile idiopathic arthritis; cancer, in particular multiple myeloma and malignant solid tumors, colorectal cancer, prostate cancer, ovarian cancer.

46. The use according to claim 44, wherein said aqueous pharmaceutical composition is administered parenterally.

47. The use according to claim 46, wherein said aqueous pharmaceutical composition is administered intramuscularly, intravenously, or subcutaneously.

48. The use of the aqueous pharmaceutical composition of levilimab according to any of claims 1, 17, 29, 30 for treating rheumatoid arthritis.

49. The use according to claim 48, wherein said aqueous pharmaceutical composition is administered at a dose of levilimab of 162 mg.

50. The use according to claim 48, wherein said aqueous pharmaceutical composition is administered once a week or once every two weeks.

51. The use according to claim 48, wherein said aqueous pharmaceutical composition is administered parenterally.

52. The use according to claim 51, wherein said aqueous pharmaceutical composition is administered intramuscularly, intravenously, or subcutaneously.

53. The use according to claim 48 further comprising the use of methotrexate.

54. The use of the aqueous pharmaceutical composition of levilimab according to any of claims 1, 17, 29, 30 for treating active rheumatoid arthritis.

55. The use according to claim 54, wherein said aqueous pharmaceutical composition is administered at a dose of levilimab of 324 mg or 648 mg.

56. The use according to claim 54, wherein said aqueous pharmaceutical composition is administered once in 2 weeks, or once in 4 weeks, or once in 6 weeks.

57. The use according to claim 54, wherein said aqueous pharmaceutical composition is administered parenterally.

58. The use according to claim 57, wherein said aqueous pharmaceutical composition is administered intramuscularly, intravenously, or subcutaneously.

59. The use according to claim 54 further comprising the use of methotrexate.

60. The use of the aqueous pharmaceutical composition of levilimab according to any of claims 1, 17, 29, 30 for treating or preventing adult (acute) respiratory distress syndrome or cytokine release syndrome.

61. The use according to claim 60, wherein said aqueous pharmaceutical composition is administered at a dose of levilimab of 324 mg or 648 mg.

62. The use according to claim 60, wherein said aqueous pharmaceutical composition is administered once, or twice, or three times, or four times at an interval of at least 8 hours.

63. The use according to claim 60, wherein said aqueous pharmaceutical composition is administered parenterally.

64. The use according to claim 63, wherein said aqueous pharmaceutical composition is administered intramuscularly, intravenously, or subcutaneously.

65. A method for producing the aqueous pharmaceutical composition according to claim 1, comprising combining 5-220 mg/ml levilimab with
0.4-1.8 mg/ml sodium acetate trihydrate;
20-50 mg/ml polyol;
5-10 mg/ml glycine; and
acetic acid to pH 4.5-6.5.

66. A method for producing the aqueous pharmaceutical composition according to claim 17, comprising combining 5-220 mg/ml levilimab with
0.4-1.8 mg/ml sodium acetate trihydrate;
10-32 mg/ml arginine hydrochloride; and
acetic acid to pH 4.5-6.5.

67. The method according to any of claims 65-66, wherein said acetic acid is glacial acetic acid.
